SEQUENCE LISTING (106 sequences)

SEQ ID NO: 1 - protein, 19 aa, Homo sapiens
VPSTPPTPSPSTPPTPSPS

SEQ ID NO: 2 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 3 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 6 aa, Homo sapiens
VPPPPP

SEQ ID NO: 5 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 6 - protein, 131 aa, Homo sapiens; identical to SEQ ID NO: 3

SEQ ID NO: 7 - protein, 58 aa, Homo sapiens
ESPKAQASSVPTAQPQAEGSLAKATTAPATTRNTGRGGEEKKKEKEKEEQEERETKTP

SEQ ID NO: 8 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 9 - protein, 117 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 10 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 11 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 12 - protein, 110 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 13 - protein, 15 aa, Homo sapiens
EPKSCDKTHTCPPCP

SEQ ID NO: 14 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 12 aa, Homo sapiens
ERKCCVECPPCP

SEQ ID NO: 17 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 18 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 20 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 21 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - protein, 12 aa, Homo sapiens
ESKYGPPCPSCP

SEQ ID NO: 23 - protein, 110 aa, Artificial Sequence
  Description of Artificial Sequence Synthetic peptide
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 24 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 26 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 27 - protein, 131 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 28 - DNA, 23 bp, Artificial Sequence
  Description of Artificial Sequence Synthetic primer
ttcttctgatcaggagcccaaat

SEQ ID NO: 29 - DNA, 24 bp, Artificial Sequence
  Description of Artificial Sequence Synthetic primer
gctcctcccgcggctttgtcttgg

SEQ ID NO: 30 - DNA, 59 bp, Artificial Sequence
  Description of Artificial Sequence Synthetic primer
ttcttctgatcaggagcccaaatcttctgacaaaactcacacatctccaccgtgcccag

SEQ ID NO: 31 - DNA, 59 bp, Artificial Sequence
  Description of Artificial Sequence Synthetic primer
gggaccgtcagtcttcctcttccccccaaaacccaaggacaccctcatgatctcccgga

SEQ ID NO: 32 - DNA, 59 bp, Artificial Sequence
  Description of Artificial Sequence Synthetic primer
tgtggtggacgtgagccacgaagaccctgaggtcaagttcaactggtacgtggacggcg

SEQ ID NO: 33 - DNA, 60 bp, Artificial Sequence
  Description of Artificial Sequence Synthetic primer
agaggaagactgacggtccaccnwncaagagttcaggtgctgggcacggtggagatgtgt

SEQ ID NO: 34 - DNA, 60 bp, Artificial Sequence
  Description of Artificial Sequence Synthetic primer
cgtggctcacgtccaccaccacgcatgtgacctcaggggtccgggagatcatgagggtgt

SEQ ID NO: 35 - DNA, 60 bp, Artificial Sequence
  Description of Artificial Sequence Synthetic primer
gctcctcccgcggctttgtcttggcattatgcacctccacgccgtccacgtaccagttga

SEQ ID NO: 36 - DNA, 59 bp, Artificial Sequence
  Description of Artificial Sequence Synthetic primer
wggaccgtcagtcttcctcttccccccaaaacccaaggacaccctcatgatctcccgga

SEQ ID NO: 37 - DNA, 60 bp, Artificial Sequence
  Description of Artificial Sequence Synthetic primer
agaggaagactgacggtccnwnacccaagagttcaggtgctgggcacggtggagatgtgt

SEQ ID NO: 38 - DNA, 59 bp, Artificial Sequence
  Description of Artificial Sequence Synthetic primer
gnwnccgtcagtcttcctcttccccccaaaacccaaggacaccctcatgatctcccgga

SEQ ID NO: 39 - DNA, 60 bp, Artificial Sequence
  Description of Artificial Sequence Synthetic primer
agaggaagactgacggnwntccacccaagagttcaggtgctgggcacggtggagatgtgt

SEQ ID NO: 40 - DNA, 59 bp, Artificial Sequence
  Description of Artificial Sequence Synthetic primer
gccgtcagtcttcctcttccccccaaaacccaaggacaccctcatgatctcccggaccc

SEQ ID NO: 41 - DNA, 59 bp, Artificial Sequence
  Description of Artificial Sequence Synthetic primer
tgtggacgtgnwnagccacgaagaccctgaggtcaagttcaactggtacgtggacggcg

SEQ ID NO: 42 - DNA, 60 bp, Artificial Sequence
  Description of Artificial Sequence Synthetic primer
ggaagaggaagactgacggtccacccaagagttcaggtgctgggcacggtggagatgtgt

SEQ ID NO: 43 - DNA, 59 bp, Artificial Sequence
  Description of Artificial Sequence Synthetic primer
cgtggctnwncacgtccaccaccacgcatgtgacctcaggggtccgggagatcatgagg

SEQ ID NO: 44 - DNA, 59 bp, Artificial Sequence
  Description of Artificial Sequence Synthetic primer
tgtggacgtgagcnwncacgaagaccctgaggtcaagttcaactggtacgtggacggcg

SEQ ID NO: 45 - DNA, 60 bp, Artificial Sequence
  Description of Artificial Sequence Synthetic primer
cgtgnwngctcacgtccaccaccacgcatgtgacctcaggggtccgggagatcatgaggg

SEQ ID NO: 46 - DNA, 33 bp, Artificial Sequence
  Description of Artificial Sequence Synthetic primer
gtctccaacaaagccnwnctcccagcccccatc

SEQ ID NO: 47 - DNA, 33 bp, Artificial Sequence
  Description of Artificial Sequence Synthetic primer
gatgggggctgggagnwnggctttgttggagac

SEQ ID NO: 48 - DNA, 33 bp, Artificial Sequence
  Description of Artificial Sequence Synthetic primer
cccaacaaagccctcnwnccagcccccatcgag

SEQ ID NO: 49 - DNA, 34 bp, Artificial Sequence
  Description of Artificial Sequence Synthetic primer
ctcgatgggggctggnwngagggctttgttggag

SEQ ID NO: 50 - DNA, 35 bp, Artificial Sequence
  Description of Artificial Sequence Synthetic primer
cacaaagccctcccanwngcccccatcgagaaaac

SEQ ID NO: 51 - DNA, 36 bp, Artificial Sequence
  Description of Artificial Sequence Synthetic primer
gttttctcgatgggggcnwntgggagggctttgttg

SEQ ID NO: 52 - DNA, 57 bp, Artificial Sequence
  Description of Artificial Sequence Synthetic primer
cagaaccacaggtgtacaccctgcccccatcccgggatgagctgaccaagaaccagg

SEQ ID NO: 53 - DNA, 57 bp, Artificial Sequence
  Description of Artificial Sequence Synthetic primer
agcttctatccaagcgacatcgccgtgcgttgggagagcaatgggcaggagctgccg

SEQ ID NO: 54 - DNA, 57 bp, Artificial Sequence
  Description of Artificial Sequence Synthetic primer
ccccgtgctggactccgacggctccttcttcctctacagcaagctcaccgtggacaa

SEQ ID NO: 55 - DNA, 57 bp, Artificial Sequence
  Description of Artificial Sequence Synthetic primer
gcttctcctgcatggtgatgcatgaggctctgccactcgccttcacgcagaagagcc

SEQ ID NO: 56 - DNA, 57 bp, Artificial Sequence
  Description of Artificial Sequence Synthetic primer
tgcttggatagaagcctttgaccaggcaggtcaggctgacctggttcttggtcagct

SEQ ID NO: 57 - DNA, 57 bp, Artificial Sequence
  Description of Artificial Sequence Synthetic primer
cgagtccagcacgggaggcgtggtcttgtagttgttctccggcagctcctgcccatt

SEQ ID NO: 58 - DNA, 57 bp, Artificial Sequence
  Description of Artificial Sequence Synthetic primer
cccatgcaggagaagacgttcccctgctgccacctgctcttgtccacggtgagcttg

SEQ ID NO: 59 - DNA, 55 bp, Artificial Sequence
  Description of Artificial Sequence Synthetic primer
cgctataatctagatcatttacccggagacagggagaggctcttctgcgtgaagg

SEQ ID NO: 60 - DNA, 26 bp, Artificial Sequence
  Description of Artificial Sequence Synthetic primer
cagaaccacaggtgtacaccctgccc

SEQ ID NO: 61 - DNA, 22 bp, Artificial Sequence
  Description of Artificial Sequence Synthetic primer
cctataatctagatcatttacc

SEQ ID NO: 62 - DNA, 58 bp, Artificial Sequence
  Description of Artificial Sequence Synthetic primer
gtcttctcctgcatggtgggccacgaggccctgccgctggccttcacacagaagacca

SEQ ID NO: 63 - DNA, 55 bp, Artificial Sequence
  Description of Artificial Sequence Synthetic primer
cgctataatctagatcatttacccgccaagcggtcgatggtcttctgtgtgaagg

SEQ ID NO: 64 - DNA, 58 bp, Artificial Sequence
  Description of Artificial Sequence Synthetic primer
aggcttctatccaagcgacatcgccgttcgctggctgcaggggtcacaggagctgccc

SEQ ID NO: 65 - DNA, 57 bp, Artificial Sequence
  Description of Artificial Sequence Synthetic primer
cgagtccagcacgggaggcgtggtcttgtacttctcgcggggcagctcctgtgaccc

SEQ ID NO: 66 - protein, 223 aa, Homo sapiens
TCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 214 aa, Homo sapiens
ACHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 68 - protein, 216 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 218 aa, Homo sapiens
PELLGGPSVFLFPPKPKDTLLLSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVRWESNGQELPENNYKTTPPVLDSDGSFFLYSKLTVDKSRWQQGNVFSCMVMHEALPLAFTQKSLSLSPGK

SEQ ID NO: 70 - protein, 218 aa, Homo sapiens
PELLGGPSVFLFPPKPKDTLLLSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVRWESNGQELPENNYKTTPPVLDSDGSFFLYSKLTVDKSRWQQGNVFSCMVGHEALPLAFTQKTIDRLAGK

SEQ ID NO: 71 - protein, 218 aa, Homo sapiens
PELLGGPSVFLFPPKPKDTLLLSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVRWLQGSQELPREKYKTTPPVLDSDGSFFLYSKLTVDKSRWQQGNVFSCMVMHEALPLAFTQKSLSLSPGK

SEQ ID NO: 72 - protein, 218 aa, Homo sapiens; identical to SEQ ID NO: 69

SEQ ID NO: 73 - protein, 209 aa, Homo sapiens
CHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDR

SEQ ID NO: 74 - protein, 209 aa, Homo sapiens
CHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDR

SEQ ID NO: 75 - protein, 212 aa, Homo sapiens
TAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDK

SEQ ID NO: 76 - protein, 208 aa, Homo sapiens
AGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSL

SEQ ID NO: 77 - protein, 208 aa, Homo sapiens
GGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSL

SEQ ID NO: 78 - protein, 208 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 79 - protein, 208 aa, Homo sapiens
  MOD_RES (80)..(80): Variable amino acid
GGPSVFLFPPKPKDTLMISRTPEVTCVVVDVSQEDPEVQFNWYVDGVEVHNAKTKPREEQFNSTYRVVSVLTVLHQDWLXGKEYKCKVSXKGLPSSIEKTISXAXGQPREPQVYTLPPSQEEMTKNQVSLTCLVKGFYPSDIAVEWESNGQPENNYKTTPPVLDSDGSFFLYSRLTVDKSXWQEGNVFSCSVMHEALHNHYTQKSLSL

SEQ ID NO: 80 - protein, 210 aa, Homo sapiens
RGVSAYLSRPSPFDLFIRKSPTITCLVVDLAPSKGTVQLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSV

SEQ ID NO: 81 - protein, 5 aa, Artificial Sequence
  Description of Artificial Sequence Synthetic peptide
LLGGP

SEQ ID NO: 82 - protein, 25 aa, Artificial Sequence
  Description of Artificial Sequence Synthetic peptide
LLXXXXXXXXXXXXXXXXXXXXGGP

SEQ ID NO: 83 - protein, 25 aa, Artificial Sequence
  Description of Artificial Sequence Synthetic peptide
LLGXXXXXXXXXXXXXXXXXXXXGP

SEQ ID NO: 84 - protein, 25 aa, Artificial Sequence
  Description of Artificial Sequence Synthetic peptide
LLGGXXXXXXXXXXXXXXXXXXXXP

SEQ ID NO: 85 - protein, 5 aa, Artificial Sequence
  Description of Artificial Sequence Synthetic peptide
DVSHE

SEQ ID NO: 86 - protein, 25 aa, Artificial Sequence
  Description of Artificial Sequence Synthetic peptide
DVXXXXXXXXXXXXXXXXXXXXSHE

SEQ ID NO: 87 - protein, 25 aa, Artificial Sequence
  Description of Artificial Sequence Synthetic peptide
DVSXXXXXXXXXXXXXXXXXXXXHE

SEQ ID NO: 88 - protein, 6 aa, Artificial Sequence
  Description of Artificial Sequence Synthetic peptide
ALPAPI

SEQ ID NO: 89 - protein, 26 aa, Artificial Sequence
  Description of Artificial Sequence Synthetic peptide
ALXXXXXXXXXXXXXXXXXXXXPAPI

SEQ ID NO: 90 - protein, 26 aa, Artificial Sequence
  Description of Artificial Sequence Synthetic peptide
ALPXXXXXXXXXXXXXXXXXXXXAPI

SEQ ID NO: 91 - protein, 26 aa, Artificial Sequence
  Description of Artificial Sequence Synthetic peptide
ALPAXXXXXXXXXXXXXXXXXXXXPI

SEQ ID NO: 92 - protein, 306 aa, Artificial Sequence
  Description of Artificial Sequence Synthetic peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXNSTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXNSTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 93 - protein, 9 aa, Artificial Sequence
  Description of Artificial Sequence Synthetic peptide
XNSTXNSTX

SEQ ID NO: 94 - protein, 6 aa, Artificial Sequence
  Description of Artificial Sequence Synthetic peptide
YPSDIA

SEQ ID NO: 95 - protein, 8 aa, Artificial Sequence
  Description of Artificial Sequence Synthetic peptide
YPNSTDIA

SEQ ID NO: 96 - protein, 9 aa, Artificial Sequence
  Description of Artificial Sequence Synthetic peptide
YNSTPSDIA

SEQ ID NO: 97 - protein, 14 aa, Artificial Sequence
  Description of Artificial Sequence Synthetic peptide
CSVMHEALHNHYTQ

SEQ ID NO: 98 - protein, 14 aa, Artificial Sequence
  Description of Artificial Sequence Synthetic peptide
CMVGHEALPLAFTQ

SEQ ID NO: 99 - protein, 4 aa, Artificial Sequence
  Description of Artificial Sequence Synthetic peptide
QPEN

SEQ ID NO: 100 - protein, 6 aa, Artificial Sequence
  Description of Artificial Sequence Synthetic peptide
QELPRE

SEQ ID NO: 101 - protein, 9 aa, Artificial Sequence
  Description of Artificial Sequence Synthetic peptide
KDTLMISRT

SEQ ID NO: 102 - protein, 9 aa, Artificial Sequence
  Description of Artificial Sequence Synthetic peptide
KDTLLISRT

SEQ ID NO: 103 - protein, 9 aa, Artificial Sequence
  Description of Artificial Sequence Synthetic peptide
DIAVEWESN

SEQ ID NO: 104 - protein, 9 aa, Artificial Sequence
  Description of Artificial Sequence Synthetic peptide
DIAVRWESN

SEQ ID NO: 105 - protein, 5 aa, Artificial Sequence
  Description of Artificial Sequence Synthetic peptide
YNSTY

SEQ ID NO: 106 - protein, 9 aa, Artificial Sequence
  Description of Artificial Sequence Synthetic peptide
YNSTYNSTY

(19) United States
(12) Patent Application Publication
(10) Pub. No.: US 2008/0227958 A1
(43) Pub. Date: Sep. 18, 2008

(54) BINDING PROTEINS COMPRISING IMMUNOGLOBULIN HINGE AND FC REGIONS HAVING ALTERED FC EFFECTOR FUNCTIONS

(75) Inventors: Peter Armstrong Thompson, Bellevue, WA (US); Erik Stephen Espling, Seattle, WA (US); Peter Robert Baum, Seattle, WA (US); Phillip Tan, Lynnwood, WA (US)

Correspondence Address: DARBY & DARBY P.C., P.O. BOX 770, Church Street Station, New York, NY, 10008-0770, US

(73) Assignee: Trubion Pharmaceuticals Inc., Seattle, WA (US)

(21) Appl. No.: 11/735,413

(22) Filed: Apr. 13, 2007

Related U.S. Application Data
(60) Provisional application No. 60/744,899, filed on Apr. 14, 2006.

(51) Int. Cl. C07K 16/00

(52) U.S. Cl. 530/387.3; 530/387.1

(57) ABSTRACT

Provided herein are binding proteins comprising one or more immunoglobulin Fc region hinge, CH2, and/or CH3 domain wherein one or more hinge and/or constant region CH2 and/or CH3 domain is modified to alter the binding protein's binding affinity and/or specificity for a cognate receptor (e.g., an Fc receptor) and/or to impart one or more new binding specificity(ies) to the hinge and/or constant region that the corresponding unmodified immunoglobulin does not possess (e.g., affinity for distinct class of cognate receptor distinct from the class of cognate receptor to which the unmodified binding protein specifically binds). Binding proteins according to the present invention include, for example, modified antibodies, antibody fragments, recombinant binding proteins, and molecularly engineered binding domain-immunoglobulin fusion proteins, including small modular immunopharmaceutical products (SMIP™ products).

### CROSS-REFERENCE TO RELATED APPLICATIONS

This application claims the benefit of U.S. Provisional Application Ser. No. 60/744,899 filed on Apr. 14, 2006, the benefit of the earlier filing date of which is hereby claimed under 35 U.S.C. §119 (e) and further incorporated by reference.

### BACKGROUND OF THE INVENTION

1. Technical Field of the Invention

The present invention relates generally to the fields of immunology, protein chemistry, and molecular biology. More specifically, provided herein are binding proteins comprising one or more immunoglobulin hinge, CH2, and/or CH3 domain wherein one or more hinge, CH2 and/or CH3 domain is modified to alter the binding protein's binding affinity and/or specificity for a cognate receptor (e.g., an Fc receptor) and/or to impart one or more new binding specificity(ies) to the hinge and/or constant region that the corresponding unmodified binding protein does not possess (e.g., affinity for an Fc receptor distinct from the cognate receptor(s) to which the unmodified binding protein specifically binds). Binding proteins according to the present invention include, for example, modified antibodies, antibody fragments, recombinant binding proteins, and molecularly engineered binding domain-immunoglobulin fusion proteins, including small modular immunopharmaceutical products (SMIP™ products).

2. Description of the Related Art

An immunoglobulin is a multimeric protein composed of two identical light chain polypeptides and two identical heavy chain polypeptides (H₂L₂) that are joined into a macromolecular complex by interchain disulfide bonds. Intrachain disulfide bonds join different areas of the same polypeptide chain resulting in the formation of loops that, along with adjacent amino acids, constitute the immunoglobulin domains.

At the amino-terminal portion, each light chain and each heavy chain has a single variable region that shows considerable variation in amino acid composition from one antibody to another. The light chain variable region, V_{L}, associates with the variable region of a heavy chain, V_{H}, to form the antigen binding site of the immunoglobulin, Fv. Light chains have a single constant region domain (CH1) and heavy chains have several constant region domains. Classes IgG, IgA, and IgD have three heavy chain constant region domains, which are designated CH1, CH2, and CH3; and the IgM and IgE classes have four heavy chain constant region domains, CH1, CH2, CH3, and CH4. Immunoglobulin structure and function are reviewed in Harlow et al., Eds., “Antibodies: A Laboratory Manual,” Chapter 14 (Cold Spring Harbor Laboratory, Cold Spring Harbor, 1988) and in Lo, Ed., “Antibody Engineering: Methods and Protocols,” Part 1 (Humana Press, Totowa, N.J., 2004).

The heavy chains of immunoglobulins can be divided into three functional regions: the Fd region, the hinge region, and the Fc region (fragment crystallizable). The Fd region comprises the V_{H} and CH1 domains and, in combination with the light chain, forms Fab—the antigen-binding fragment. The Fc fragment is responsible for the immunoglobulin effector functions, which include, for example, complement fixation and binding to cognate Fc receptors of effector cells. The hinge region, found in IgG, IgA, and IgD immunoglobulin classes, acts as a flexible spacer that allows the Fab portion to move freely in space relative to the Fc region. In contrast to the constant regions, the hinge domains are structurally diverse, varying in both sequence and length among immunoglobulin classes and subclasses.

According to crystallographic studies, the immunoglobulin hinge region can be further subdivided structurally and functionally into three regions: the upper hinge, the core, and the lower hinge. Shin et al., Immunological Reviews 130:87 (1992). The upper hinge includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges. The lower hinge region joins the amino terminal end of, and includes residues in, the CH2 domain. Id.

The core hinge region of human IgG₁ contains the sequence Cys-Pro-Pro-Cys that, when dimerized by disulfide bond formation, results in a cyclic octapeptide believed to act as a pivot, thus conferring flexibility. Conformational changes permitted by the structure and flexibility of the immunoglobulin hinge region polypeptide sequence may affect the effector functions of the Fc portion of the antibody.

Three general categories of effector functions associated with the Fc region include (1) activation of the classical complement cascade, (2) interaction with effector cells, and (3) compartmentalization of immunoglobulins. The different human IgG subclasses vary in the relative efficacies with which they fix complement, or activate and amplify the steps of the complement cascade (see, e.g., Kirschfink, Immunol. Rev. 180:177 (2001); Chakraborti et al. Cell Signal 12:607 (2000); Kohl et al., Mol. Immunol. 36:893 (1999); Marsh et al., Curr. Opin. Nephrol. Hypertens. 8:557 (1999); and Speth et al., Wien Klin. Wochenschr. 111:378 (1999)). Complement-dependent cytotoxicity (CDC) is believed to be a significant mechanism for clearance of specific target cells such as tumor cells. In general, IgG₁ and IgG₃ most effectively fix complement, IgG₂ is less effective, and IgG₄ does not activate complement. Complement activation is initiated by binding of C1q, a subunit of the first component C1 in the cascade, to an antigen-antibody complex.

Even though the binding site for C1q is located in the CH2 domain of the antibody, the hinge region influences the ability of the antibody to activate the cascade. For example, recombinant immunoglobulins lacking a hinge region are unable to activate complement. Shin et al., (1992), supra. Without the flexibility conferred by the hinge region, the Fab portion of the antibody bound to the antigen may not be able to adopt the conformation required to permit C1q to bind to CH2. (See Id.). Hinge length and segmental flexibility correlate, to a limited extent, with complement activation. Thus, human IgG₃ molecules with altered hinge regions that are as rigid as IgG₄ hinge regions remain effective in activation of the complement cascade.

The hinge region may also contain one or more glycosylation site(s), which include a number of structurally distinct types of sites for carbohydrate attachment. For example, IgA₁ contains five glycosylation sites within a 17 amino acid segment of the hinge region, conferring exceptional resistance of the hinge region polypeptide to intestinal proteases, considered an advantageous property for a secretory immunoglobulin.

The absence of a hinge region, or lack of a functional hinge region, can also affect the ability of certain human immunoglobulins to bind Fc receptors on immune effector cells. Binding of an immunoglobulin to an Fc receptor facilitates antibody-dependent cell-mediated cytotoxicity (ADCC), which is presumed to be an important mechanism for the elimination of tumor cells. The human IgG Fc receptor (FcR) family is divided into three groups, FcγRI (CD64), which is capable of binding IgG with high affinity, and FcγRII (CD32) and FcγRIII (CD16), both of which are low affinity receptors. The human IgA Fc receptor is FcαR (CD89). Experimental evidence indicates that residues in the hinge proximal region of the CH2 domain are important to the specificity of the interaction between an immunoglobulin and each of the respective Fc receptors. This is supported by the observation that IgG₁ myeloma proteins and recombinant IgG₃ chimeric antibodies that lack a hinge region are unable to bind FcγRI, likely owing to decreased accessibility to the CH2 domain. Shin et al., Intern. Rev. Immunol 10:177, 178-79 (1993). Fcγ-receptors are reviewed generally in Ingmar et al., “Human IgG Fc Receptors,” Intern. Rev. Immunol. 16:29-55 (1997) and Ravetch and Bolland, “IgG Fc Receptors,” Annu. Rev. Immunol. 19:275-90 (2001). See, also, Getahun et al., J. Immunol. 172:5269-5276 (2004).

FcγRI (CD64) is expressed on macrophages and dendritic cells and plays a role in phagocytosis, respiratory burst, cytokine stimulation, and dendritic cell endocytic transport. Expression of FcγRI is upregulated by both GM-CSF and γ-interferon (γ-IFN) and downregulated by interleukin-4 (IL-4). When all activating receptors are knocked out, mice are protected from immune complex mediated inflammation. Similarly, when FCγRI is knocked out, mice are afforded some protection.

Three forms of FcγRII (CD32) have been identified, FcγRIIa, FcγRIIb, and FcγRIIc. FcγRIIa is expressed on polymorphonuclear leukocytes (PMN), macrophages, dendritic cells, and mast cells. FcγRIIa plays a role in phagocytosis, respiratory burst, and cytokine stimulation. Expression of FcγRIIa is upregulated by GM-CSF and γ-IFN, and decreased by IL-4. When all activating receptors are knocked out, mice are protected from immune complex mediated inflammation. FcγRIIa binds c-reactive protein (CRP) polymorph H131 with high affinity and CRP polymorph R131 with low affinity. The distribution of polymorphisms in the general population is approximately 50:50 R131 associated with an increased susceptibility to infection and lupus nephritis. FcγIIb is expressed on B cells, PMN, macrophages, and mast cells. FcγIIb inhibits immunoreceptor tyrosine-based activation motif (ITAM) mediated responses; thus, this is an inhibitory receptor. Expression of FcγRIIc is upregulated by intravenous immunoglobulin (IVIG) and IL-4 and decreased by γ-IFN. FcγRIIb knockout mice exhibit increased antibody responses and susceptibility to autoimmune disease and diminished B cell recall responses when follicular dendritic cells (FDC) are knocked-out for CD32. FcγRIIc is expressed on NK cells but its function and regulation of expression are poorly understood.

Two forms of FcγRIII (CD16) have been identified, FcγRIIIa and FcγRIIIb. FcγRIIIa is expressed on natural killer (NK) cells, macrophages, mast cells, and platelets. This receptor participates in phagocytosis, respiratory burst, cytokine stimulation, platelet aggregation and degranulation, and NK-mediated ADCC. Expression of FcγRIII is upregulated by C5a, TGFβ, and γ-IFN and downregulated by IL-4. When all activating receptors are knocked-out, mice are protected from immune complex mediated inflammation. FcγRIIa is polymorphic with F176 being the most common and V-176 being less common. F-176 binds with less avidity to IgG and is associated with lupus erythematosus. FcγRIIIb is a GPI linked receptor expressed on PMN. An inherited deficiency of FcγRIIIb exists and has no known phenotype. An FcγIIIb NA1/NA2 polymorphism is important in isoimmune neutropenia.

Monoclonal antibody technology and genetic engineering methods have led to rapid development of immunoglobulin molecules for diagnosis and treatment of human diseases. Protein engineering has been applied to improve the affinity of an antibody for its cognate antigen, to diminish problems related to immunogenicity of administered recombinant polypeptides, and to alter antibody effector functions. The domain structure of immunoglobulins is amenable to recombinant engineering, in that the antigen binding domains and the domains conferring effector functions may be exchanged between immunoglobulin classes (e.g., IgG, IgA, and IgE) and subclasses (e.g., IgG₁, IgG₂, IgG₃, and IgG₄, etc.).

In addition, smaller immunoglobulin molecules have been constructed to overcome problems associated with whole immunoglobulin therapy. For instance, single chain immunoglobulin variable region fragment polypeptides (scFv) comprise an immunoglobulin heavy chain variable domain joined via a short linker peptide to an immunoglobulin light chain variable domain. Huston et al. Proc. Natl. Acad. Sci. USA, 85:5879-83 (1988). Because of the small size of scFv molecules, they exhibit very rapid clearance from plasma and tissues and are capable of more effective penetration into tissues than whole immunoglobulins. See, e.g., Jain, Cancer Res. 50:814s-819s (1990) An anti-tumor scFv showed more rapid tumor penetration and more even distribution through the tumor mass than the corresponding chimeric antibody. Yokota et al., Cancer Res. 52:3402-08 (1992). Fusion of an scFv to another molecule, such as a toxin, takes advantage of the specific antigen-binding activity and the small size of an scFv to deliver the toxin to a target tissue. Chaudary et al., Nature 339:394 (1989) and Batra et al., Mol. Cell. Biol. 11:2200 (1991).

Despite the advantages that scFv molecules bring to serotherapy, several drawbacks to this therapeutic approach exist. While rapid clearance of scFv may reduce toxic effects in normal cells, such rapid clearance may prevent delivery of a minimum effective dose to the target tissue. Manufacturing adequate amounts of scFv for administration to patients has been challenging due to difficulties in expression and isolation of scFv that adversely affect the yield. During expression, scFv molecules lack stability and often aggregate due to pairing of variable regions from different molecules. Furthermore, production levels of scFv molecules in mammalian expression systems are low, limiting the potential for efficient manufacturing of scFv molecules for therapy. Davis et al., J. Biol. Chem. 265:10410-18 (1990) and Traunecker et al., EMBO J. 10:3655-59 (1991). Strategies for improving production have been explored, including addition of glycosylation sites to the variable regions. See, e.g., U.S. Pat. No. 5,888,773 and Jost et al., J. Biol. Chem. 269:26267-73 (1994).

An additional disadvantage to using scFv for therapy is the lack of effector functions. An scFv that lacks the cytolytic functions, ADCC and complement dependent-cytotoxicity (CDC), which are typically associated with immunoglobulin constant regions, may be ineffective for treating disease. Even though development of scFv technology began nearly two decades ago, currently no scFv products are approved for therapy. Conjugation or fusion of toxins to scFV has thus been an alternative strategy to provide a potent, antigen-specific molecule, but dosing with such conjugates or chimeras is often limited by excessive and/or non-specific toxicity having its origin in the toxin moiety of such preparations. Toxic effects may include supraphysiological elevation of liver enzymes and vascular leak syndrome, and other undesired effects. In addition, immunotoxins are themselves highly immunogenic after being administered to a host, and host antibodies generated against the immunotoxin limit its potential usefulness in repeated therapeutic treatments of an individual.

The benefits of immunoglobulin constant region-associated effector functions in the treatment of disease has prompted development of fusion proteins in which immunoglobulin constant region polypeptide sequences are present and non-immunoglobulin sequences are substituted for the antibody variable region. For example, CD4, the T cell surface protein recognized by HIV, was recombinantly fused to an immunoglobulin Fc effector domain. See, Sensel et al., Chem. Immunol. 65:129-158 (1997). The biological activity of such a molecule depends, in part, on the class or subclass of the constant region chosen. An IL-2-IgG₁ fusion protein, for example, effects complement-mediated lysis of IL-2 receptor-bearing cells. See Id. Use of immunoglobulin constant regions to construct these and other fusion proteins may also confer improved pharmacokinetic properties.

There remains an unmet need in the art for improved immunoglobulin-derived binding proteins wherein hinge and/or Fc domains are modified to alter one or more effector function(s) such as altered Fc receptor binding affinity and/or specificity (and associated ADCC), binding-protein in vivo half-life, and/or complement fixation.

### SUMMARY OF THE INVENTION

The present invention addresses these and other related needs by providing, inter alia, binding proteins comprising one or more immunoglobulin constant region hinge, CH2, and/or CH3 domain(s) wherein one or more hinge and/or constant region CH2 and/or CH3 domain is modified to alter one or more of the binding protein's Fc effector function(s). Exemplified herein are binding proteins wherein the immunoglobulin hinge and/or Fc region is modified to achieve an altered binding affinity and/or specificity for a cognate receptor (e.g., an Fc receptor) and/or to impart one or more new binding specificity(ies) to the Fc region that the corresponding unmodified binding protein does not possess (e.g., affinity for one or more Fc receptor that is distinct from the cognate receptor to which the unmodified binding protein specifically binds).

Binding proteins according to the present invention include, for example, modified antibodies, antibody fragments, recombinant binding proteins, and molecularly engineered binding domain-immunoglobulin fusion proteins, including small modular immunopharmaceutical products (SMIP™ products) wherein one or more amino acid sequence(s) in an immunoglobulin hinge, CH2, and/or CH3 domain is altered. Within certain embodiments, the modified binding proteins disclosed herein comprise changes in one or more amino acid sequence(s) in the hinge, CH2, and/or CH3 domain that are responsible for receptor binding affinity and/or specificity.

In one aspect, the present invention provides binding proteins, in particular binding proteins comprising one or more immunoglobulin heavy chain hinge, CH2, and/or CH3 domain, wherein the binding protein is modified such that it binds with altered (i.e. either increased or decreased) binding affinity and/or specificity to one or more immunoglobulin-specific Fc receptor.

Exemplified herein are binding proteins comprising one or more IgG immunoglobulin heavy chain hinge, CH2, and/or CH3 domain, wherein the binding protein is modified such that it binds with altered (i.e. either increased or decreased) binding affinity and/or specificity to one or more of the IgG immunoglobulin-specific Fc receptors FcγRI (CD64); FcγRII (CD32), including FcγRIIa, FcγRIIb, and FcγRIIc; and/or FcγRIII (CD16), including FcγRIIIa and FcγRIIIb. Binding proteins of this type include, for example, binding proteins wherein one or more amino acid(s) is inserted into and/or deleted from the primary amino acid sequence in regions, domains, turns, and/or loop structures responsible for Fcγ-receptor binding. Such changes include, but are not limited to, the insertion and/or deletion of one or more amino acid(s) between and/or adjacent to amino acids that, upon binding to a cognate Fc receptor, are in direct contact with amino acids within one or more immunoglobulin-specific Fc receptor(s) including FcγRI (CD64), FcγRII (CD32), and/or FcγRIII (CD16).

Specific embodiments of these aspects of the present invention include binding proteins comprising one or more IgG CH2 domain wherein the CH2 domain is an IgG, and/or an IgG₃ CH2 domain. Some such embodiments provide binding proteins comprising one or more amino acid deletion from and/or amino acid insertion within the hinge proximal loop structure, L-L-G-G-P, of the IgG₁ and/or IgG₃ CH2 domain. Specifically exemplified herein are binding proteins comprising single insertions of a single amino acid at the positions indicated by the “*” within the following hinge proximal loop structure. Thus, provided herein are binding proteins comprising the modified hinge proximal loop structures L-L-*-G-G-P, L-L-G-*-G-P, and L-L-G-G-*-P. Also provided herein are binding proteins comprising single insertions of two or more amino acids at the positions indicated by “*” within the hinge proximal loop structures L-L-*-G-G-P, L-L-G-*-G-P, and L-L-G-G-*-P. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified hinge proximal loop structures are selected from the group consisting of Ala, Gly, Ile, Leu, and Val.

Other such embodiments provide binding proteins comprising one or more IgG CH2 domain wherein the CH2 domain is an IgG₁, IgG₂, and/or IgG₃ CH2 domain. Some such embodiments provide binding proteins comprising one or more amino acid deletion from and/or amino acid insertion within the BC loop structure, D-V-S-H-E, of the IgG₁, IgG₂, and/or IgG₃ CH2 domain. Specifically exemplified herein are binding proteins comprising single insertions of a single amino acid at the positions indicated by the “*” within the following BC loop structure. Thus, provided herein are binding proteins comprising the modified BC loop structures D-V-*-S-H-E and D-V-S-*-H-E. Also provided herein are binding proteins comprising single insertions of two or more amino acids at the positions indicated by “*” within the BC loop structures D-V-*-S-H-E and D-V-S-*-H-E. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified BC loop structures are selected from the group consisting of Ala, Gly, Ile, Leu, and Val.

Still other such embodiments provide binding proteins comprising one or more IgG CH2 domain wherein the CH2 domain is an IgG₁ and/or IgG₃ CH2 domain. Some such embodiments provide binding proteins comprising one or more amino acid deletion from and/or amino acid insertion within the FG loop structure, A-L-P-A-P-I, of the CH2 domain. Specifically exemplified herein are binding proteins comprising single insertions of a single amino acid at the positions indicated by the “*” within the following FG loop structure. Thus, provided herein are binding proteins comprising the modified FG loop structures A-L-*-P-A-P-I, A-L-P-*-A-P-I, and A-L-P-A-*-P-I. Also provided herein are binding proteins comprising single insertions of two or more amino acids at the positions indicated by “*” within the FG loop structures A-L-*-P-A-P-I, A-L-P-*-A-P-I, and A-L-P-A-*-P-I. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified FG loop structures are selected from the group consisting of Ala, Gly, Ile, Leu, and Val.

In another aspect, the present invention provides binding proteins, in particular binding proteins comprising one or more heavy chain hinge, CH2, and/or CH3 domain, wherein the binding protein comprises one or more modification(s) within the one or more heavy chain hinge, CH2, and/or CH3 domain wherein the modification comprises the insertion of one or more N-linked glycosylation sequence(s) and/or one or more O-linked glycosylation sequence(s), which glycosylation sequence(s) is sufficient to achieve N- and/or O-linked glycosylation at the position of insertion thereby altering (i.e. either increasing or decreasing) the binding protein's binding affinity and/or specificity to one or more immunoglobulin-specific Fc receptor. Binding proteins of this type include, for example, those comprising changes in the primary amino acid sequence at positions that are proximal and/or distal to regions, domains, and/or loop structures responsible for glycosylation in the unmodified binding protein.

Exemplified herein are binding proteins comprising one or more IgG heavy chain hinge, CH2, and/or CH3 domain, wherein the binding proteins comprise one or more modification(s) within the one or more IgG heavy chain hinge, CH2, and/or CH3 domain wherein the modification comprises the insertion of one or more N-linked glycosylation sequence(s) and/or one or more O-linked glycosylation sequence(s), which glycosylation sequence(s) is sufficient to achieve N- and/or O-linked glycosylation at the position of insertion thereby altering (i.e. either increasing or decreasing) the binding protein's binding affinity and/or specificity to one or more IgG immunoglobulin-specific receptor(s) FcγRI (CD64); FcγRII (CD32), including FcγRIIa, FcγRIIb, and FcγRIIc; and/or FcγRIII (CD16), including FcγRIIIa and FcγRIIIb, as compared to a corresponding binding protein comprising one or more unmodified IgG heavy chain hinge, CH2, and/or CH3 domain.

Specific embodiments of these aspects of the present invention include binding proteins comprising one or more IgG hinge domain, one or more IgG CH2 domain, and/or one or more IgG CH3 domain wherein the hinge, CH2, and/or CH3 domain is an IgG₁ hinge, CH2, and/or CH3 domain, an IgG₂ hinge, CH2, and/or CH3 domain, an IgG₃ hinge, CH2, and/or CH3 domain, and/or an IgG₄ hinge, CH2, and/or CH3 domain. Some such embodiments provide binding proteins comprising the insertion of one or more N-linked glycosylation sequence N-X-(S/T) (wherein X is any amino acid) and/or one or more O-linked glycosylation sequence X-P-X-X (wherein at least one X is T), T-X-X-X (wherein at least one X is T), X-X-T-X (wherein at least one X is R or K), and/or S-X-X-X (wherein at least one X is S)) proximal to and/or distal to the site of N-linked and/or O-linked glycosylation in the corresponding native IgG immunoglobulin hinge, CH2, and/or CH3 domain. Within certain aspects of these embodiments, the binding protein exhibits an altered (i.e. an increased or decreased) FcγR binding affinity and/or specificity.

Exemplified herein are such embodiments wherein the binding proteins comprise one or more IgG hinge domain, one or more IgG CH2 domain, and/or one or more IgG CH3 domain and wherein the binding proteins further comprise an insertion of one or more N-linked glycosylation sequence N-X-(S/T) (wherein X is any amino acid). For example, the present invention provides such binding proteins comprising an insertion of one or more N-X-(S/T) sequence adjacent to the native N-S-T sequence within the DE loop of one or more IgG₁, IgG₂, IgG₃, and/or IgG₄ CH2 domain. Within certain aspects of these embodiments, the binding protein exhibits an altered (i.e. an increased or decreased) FcγR binding affinity and/or specificity.

Within specific such embodiments, the N-linked glycosylation sequence within the DE loop of one or more IgG₁, IgG₂, IgG₃, and/or IgG₄ CH2 domain comprises the amino acid sequence N-S-T and is inserted adjacent to and/or within 0 to 100 amino acids amino-terminal and/or carboxy-terminal to the native N-S-T sequence such that the native amino acid sequence X-N-S-T-Z is modified to (AA^{a})-N-S-T-(AA^{b})-N-S-T-(AA^{c}) wherein each of AA^{a} AA^{b}, and AA^{c} independently designate from 1 to 100 amino acids. Within specific such embodiments, the N-linked glycosylation sequence within the DE loop of one or more IgG₁, IgG₂, IgG₃, and/or IgG₄ CH2 domain comprises the amino acid sequence N-S-T and is inserted adjacent to the native N-S-T sequence such that the native amino acid sequence X-N-S-T-Z is modified to X-N-S-T-Z-N-S-T-Z, wherein X and Z are independently selected from Tyr (Y) and Phe (F).

Within alternative such embodiments, the N-linked glycosylation sequence inserted within the BC loop of one or more IgG₁, IgG₂, IgG₃, and/or IgG₄ CH3 domain comprises the amino acid sequence N-S-T and is inserted distal to the native N-S-T sequence such that the native amino acid sequence Y-P-S-D-I-A is modified to Y-P-N-S-T-D-I-A and Y-N-S-T-P-S-D-I-A.

Within still further aspects, the present invention provides binding proteins, in particular binding proteins comprising one or more heavy chain hinge, CH2, and/or CH3 domain of a first immunoglobulin class (i.e. IgA, IgD, IgE, IgG, or IgM), wherein the binding protein is modified (i.e. by amino acid replacement and/or amino acid insertion) in the primary amino sequence of one or more heavy chain hinge, CH2, and/or CH3 domain of the first immunoglobulin class to generate a binding protein capable of binding to one or more cognate Fc receptor of a second immunoglobulin class distinct from the first immunoglobulin class. Such changes include, for example, replacing and/or remodeling one or more loops, or amino acid and/or peptide portions thereof, of a first immunoglobulin domain with one or more loops, or amino acid and/or peptide portions thereof, of a second immunoglobulin domain, wherein the second immunoglobulin domain comprises one or more amino acids that form at least a portion of a binding sequence for a second immunoglobulin-specific Fc receptor. Binding proteins according to these aspects of the present invention are capable of specifically binding to FcαR in addition to being capable of specifically binding to FcγRI, FcγRII, and/or FcγRIII.

Exemplified herein are binding proteins comprising one or more IgG heavy chain hinge, CH2, and/or CH3 domain, wherein the binding protein is modified to bind to one or more non-IgG immunoglobulin-specific Fc receptor including, but not limited to, the IgA immunoglobulin-specific receptor FcαR (CD89). Binding proteins of this type include, for example, binding proteins comprising changes (i.e. amino acid replacement and/or amino acid insertion) in the primary amino acid sequence of one or more IgG heavy chain hinge, CH2, and/or CH3 domain to generate amino acid sequences capable of non-IgG immunoglobulin-specific Fc receptor binding such as, for example, Fcα-receptor binding.

Within certain such embodiments are provided binding proteins comprising one or more IgG heavy chain hinge, CH2, and/or CH3 domain, wherein the binding protein is modified to bind to the IgA immunoglobulin-specific receptor FcαR (CD89). Such exemplary binding protein comprises one or more amino acid substitution(s) within the IgG CH3 FG loop and/or one or more amino acid substitution(s) within the IgG CH3 CD loop. For example, one such exemplary binding protein comprises the replacement of the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof.

Another exemplary binding protein comprises the replacement of the IgG CH3 CD loop comprising the amino acid sequence Q-P-E-N, or a portion thereof, with the IgA CH3 CD loop comprising the amino acid sequence Q-E-L-P-R-E, or a portion thereof. Yet another such exemplary binding protein comprises the replacement of both the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof, and the IgG CH3 CD loop comprising the amino acid sequence Q-P-E-N, or a portion thereof, with the IgA CH3 CD loop comprising the amino acid sequence Q-E-L-P-R-E, or a portion thereof.

Any of the aforementioned binding protein embodiments may further comprise the substitution of IgG heavy chain CH3 amino acid Met (at CH3 amino acid position no. 28 within the sequence K-D-T-L-M-I-S-R-T) with amino acid Leu such that the binding protein further comprises the amino acid sequence K-D-T-L-L-I-S-R-T. Alternatively or additionally, any of the aforementioned binding protein embodiments may further comprise the substitution of IgG heavy chain CH3 amino acid Glu (at CH3 amino acid position no. 157 within the sequence D-I-A-V-E-W-E-S-N) with amino acid Arg such that the binding protein further comprises the amino acid sequence D-I-A-V-R-W-E-S-N.

These and other aspects of the present invention will become apparent upon reference to the following detailed description and attached drawings. All publications, patents, and patent applications cited herein, whether supra or infra, are hereby incorporated by reference in their entirety.

### BRIEF DESCRIPTION OF THE FIGURES AND SEQUENCE IDENTIFIERS

FIG. 1 presents the alignment of IgG and IgA CH2 domains.

FIG. 2 presents the alignment of a wild-type IgG CH2-CH3 region and three exemplary modifications to this region suitable for generating binding proteins according to the present invention.

FIG. 3 presents the alignment of CH2 and CH2 regions from IgA1, IgA2, IgM, IgG2, IgG2, IgG2, IgG2, and IgE and the corresponding immunoglobulin loops. (From Herr et al., Nature 423:614-620 (2003))

SEQ ID NO: 1 is the amino acid sequence of human IgA₁ hinge region (VPSTPPTPSPSTPPTPSPS).

SEQ ID NO: 2 is the amino acid sequence of human IgA₁ CH2 region (CCHPRLSLHRPALEDLLLGSEANLTCTLTGLRDASGVTFTWTPSSGKSAVQGPPE RDLCGCYSVSSVLPGCAEPWNHGKTFTCTAAYPESKTPLTATLSKS)

SEQ ID NO: 3 is the amino acid sequence of human IgA₁ CH3 region (GNTFRPEVHLLPPPSEELALNELVTLTCLARGFSPKDVLVRWLQGSQELPREKYL TWASRQEPSQGTTTFAVTSILRVAAEDWKKGDTFSCMVGHEALPLAFTQKTIDR LAGKPTHVNVSVVMAEVDGTCY).

SEQ ID NO: 4 is the amino acid sequence of human IgA₂ hinge region (VPPPPP).

SEQ ID NO: 5 is the amino acid sequence of human IgA₂CH2 region (CCHPRLSLHRPALEDLLLGSEANLTCTLTGLRDASGATFTWTPSSGKSAVQGPPE RDLCGCYSVSSVLPGCAQPWNHGETFTCTAAHPELKTPLTANITKS).

SEQ ID NO: 6 is the amino acid sequence of human IgA₂ CH3 region (GNTFRPEVHLLPPPSEELALNELVTLTCLARGFSPKDVLVRWLQGSQELPREKYL TWASRQEPSQGTTTFAVTSILRVAAEDWKKGDTFSCMVGHEALPLAFTQKTIDR LAGKPTHVNVSVVMAEVDGTCY).

SEQ ID NO: 7 is the amino acid sequence of human IgD hinge region (ESPKAQASSVPTAQPQAEGSLAKATTAPATTRNTGRGGEEKKKEKEKEEQEERE TKTP).

SEQ ID NO: 8 is the amino acid sequence of human IgD CH2 region (ECPSHTQPLGVYLLTPAVQDLWLRDKATFTCFVVGSDLKDAHLTWEVAGKVPT GGVEEGLLERHSNGSQSQHSRLTLPRSLWNAGTSVTCTLNHPSLPPQRLMALREP SEQ ID NO: 9 is the amino acid sequence of human IgD CH3 region (AAQAPVKLSLNLLASSDPPEAASWLLCEVSGFSPPNILLMWLEDQREVNTSGFA PARPPPQPRSTTFWAWSVLRVPAPPSPQPATYTCVVSHEDSRTLLNASRSLEVSY VTDHGPMK).

SEQ ID NO: 10 is the amino acid sequence of human IgE CH2 region (VCSRDFTPPTVKILQSSCDGGGHFPPTIQLLCLVSGYTPGTINITWLEDGQVMDV DLSTASTTQEGELASTQSELTLSQKHWLSDRTYTCQVTYQGHTFEDSTKKCA).

SEQ ID NO: 11 is the amino acid sequence of human IgE CH3 region (DSNPRGVSAYLSRPSPFDLFIRKSPTITCLVVDLAPSKGTVNLTWSRASGKPVNH STRKEEKQRNGTLTVTSTLPVGTRDWIEGETYQCRVTHPHLPRALMRSTTKTS).

SEQ ID NO: 12 is the amino acid sequence of human IgE CH4 region (GPRAAPEVYAFATPEWPGSRDKRTLACLIQNFMPEDISVQWLHNEVQLPDARHS TTQPRKTKGSGFFVFSRLEVTRAEWEQKDEFICRAVHEAASPSQTVQRAVSVNP GK).

SEQ ID NO: 13 is the amino acid sequence of human IgG₁ hinge region (EPKSCDKTHTCPPCP).

SEQ ID NO: 14 is the amino acid sequence of human IgG₁ CH2 region (APELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISK AK).

SEQ ID NO: 15 is the amino acid sequence of human IgG₁ CH3 region (GQPREPQVYTLPPSRDELTKNQVSLTCLVKGFYPSDIAVEWESNGQPENNYKTT PPVLDSDGSFFLYSKLTVDKSRWQQGNVFSCSVMHEALHNHYTQKSLSLSPGK).

SEQ ID NO: 16 is the amino acid sequence of human IgG₂ hinge region (ERKCCVECPPCP).

SEQ ID NO: 17 is the amino acid sequence of human IgG₂ CH2 region (APPVAGPSVFLFPPKPKDTLMISRTPEVTCVVVDVSHEDPEVQFNWYVDGVEVH NAKTKPREEQFNSTFRVVSVLTVVHQDWLNGKEYKCKVSNKGLPAPIEKTISKT K).

SEQ ID NO: 18 is the amino acid sequence of human IgG₂ CH3 region (GQPREPQVYTLPPSREEMTKNQVSLTCLVKGFYPSDIAVEWESNGQPENNYKTT PPMLDSDGSFFLYSKLTVDKSRWQQGNVFSCSVMHEALHNHYTQKSLSLSPGK).

SEQ ID NO: 19 is the amino acid sequence of human IgG₃ hinge region (ELKTPLGDTTHTCPRCPEPKSCDTPPPCPRCPEPKSCDTPPPCPRCPEPKSCDTPPP CPRCP).

SEQ ID NO: 20 is the amino acid sequence of human IgG₃ CH2 region (APELLGGPSVFLFPPKPKDTLMISRTPEVTCVVVDVSHEDPEVQFKWYVDGVEV HNAKTKPREEQYNSTFRVVSVLTVLHQDWLNGKEYKCKVSNKALPAPIEKTISK TK).

SEQ ID NO: 21 is the amino acid sequence of human IgG₃ CH3 region (GQPREPQVYTLPPSREEMTKNQVSLTCLVKGFYPSDIAVEWESSGQPENNYNTT PPMLDSDGSFFLYSKLTVDKSRWQQGNIFSCSVMHEALHNRFTQKSLSLSPGK).

SEQ ID NO: 22 is the amino acid sequence of human IgG₄ hinge region (ESKYGPPCPSCP).

SEQ ID NO: 23 is the amino acid sequence of human IgG₄ CH2 region (APEFLGGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPS SIEKTISK AK).

SEQ ID NO: 24 is the amino acid sequence of human IgG₄ CH3 region (GQPREPQVYTLPPSQEEMTKNQVSLTCLVKGFYPSDIAVEWESNGQPENNYKTT PPVLDSDGSFFLYSRLTVDKSRWQEGNVFSCSVMHEALHNHYTQKSLSLSLGK).

SEQ ID NO: 25 is the amino acid sequence of human IgM CH2 region (VIAELPPKVSVFVPPRDGFFGNPRKSKLICQATGFSPRQIQVSWLREGKQVGSGV TTDQVQAEAKESGPTTYKVTSTLTIKESDWLGQSMFTCRVDHRGLTFQQNASSM CVP).

SEQ ID NO: 26 is the amino acid sequence of human IgM CH3 region (DQDTAIRVFAIPPSFASIFLTKSTKLTCLVTDLTTYDSVTISWTRQNGEAVKTHTN ISESHPNATFSAVGEASICEDDWNSGERFTCTVTHTDLPSPLKQTISRPK).

SEQ ID NO: 27 is the amino acid sequence of human IgM CH4 region (GVALHRPDVYLLPPAREQLNLRESATITCLVTGFSPADVFVQWMQRGQPLSPEK YVTSAPMPEPQAPGRYFAHSILTVSEEEWNTGETYTCVVAHEALPNRVTERTVD KSTGKPTLYNVSLVMSDTAGTCY).

SEQ ID NO: 28 is the nucleotide sequence of the oligonucleotide primer designated herein as Bci-I Forward (ttc ttc tga tca gga gcc caa at).

SEQ ID NO: 29 is the nucleotide sequence of the oligonucleotide primer designated herein as Sac-II Reverse (GCT CCT CCC GCG GCT TTG TCT TGG).

SEQ ID NO: 30 is the nucleotide sequence of the oligonucleotide primer designated herein as Lib1A_F1 (ttc ttc tga tca gga gcc caa atc ttc tga caa aac tca cac atc tcc acc gtg ccc ag).

SEQ ID NO: 31 is the nucleotide sequence of the oligonucleotide primer designated herein as Lib1A_F2 (ggg acc gtc agt ctt cct ctt ccc ccc aaa acc caa gga cac cct cat gat ctc ccg ga).

SEQ ID NO: 32 is the nucleotide sequence of the oligonucleotide primer designated herein as Lib1A_F3 (tgt ggt gga cgt gag cca cga aga ccc tga ggt caa gtt caa ctg gta cgt gga cgg cg).

SEQ ID NO: 33 is the nucleotide sequence of the oligonucleotide primer designated herein as Lib1A_R1 (AGA GGA AGA CTG ACG GTC CAC CNW NCA AGA GTT CAG GTG CTG GGC ACG GTG GAG ATG TGT).

SEQ ID NO: 34 is the nucleotide sequence of the oligonucleotide primer designated herein as Lib1A_R2 (CGT GGC TCA CGT CCA CCA CCA CGC ATG TGA CCT CAG GGG TCC GGG AGA TCA TGA GGG TGT).

SEQ ID NO: 35 is the nucleotide sequence of the oligonucleotide primer designated herein as Lib1A_R3 (GCT CCT CCC GCG GCT TTG TCT TGG CAT TAT GCA CCT CCA CGC CGT CCA CGT ACC AGT TGA).

SEQ ID NO: 36 is the nucleotide sequence of the oligonucleotide primer designated herein as Lib1B_F2 (wgg acc gtc agt ctt cct ctt ccc ccc aaa acc caa gga cac cct cat gat ctc ccg ga).

SEQ ID NO: 37 is the nucleotide sequence of the oligonucleotide primer designated herein as Lib1B_R1 (AGA GGA AGA CTG ACG GTC CNW NAC CCA AGA GTT CAG GTG CTG GGC ACG GTG GAG ATG TGT).

SEQ ID NO: 38 is the nucleotide sequence of the oligonucleotide primer designated herein as Lib1C_F2 (gnw ncc gtc agt ctt cct ctt ccc ccc aaa acc caa gga cac cct cat gat ctc ccg ga).

SEQ ID NO: 39 is the nucleotide sequence of the oligonucleotide primer designated herein as LiblC_R1 (AGA GGA AGA CTG ACG GNW NTC CAC CCA AGA GTT CAG GTG CTG GGC ACG GTG GAG ATG TGT).

SEQ ID NO: 40 is the nucleotide sequence of the oligonucleotide primer designated herein as Lib2A_F2 (gcc gtc agt ctt cct ctt ccc ccc aaa acc caa gga cac cct cat gat ctc ccg gac cc).

SEQ ID NO: 41 is the nucleotide sequence of the oligonucleotide primer designated herein as Lib2A_F3 (tgt gga cgt gnw nag cca cga aga ccc tga ggt caa gtt caa ctg gta cgt gga cgg cg).

SEQ ID NO: 42 is the nucleotide sequence of the oligonucleotide primer designated herein as Lib2A_R1 (GGA AGA GGA AGA CTG ACG GTC CAC CCA AGA GTT CAG GTG CTG GGC ACG GTG GAG ATG TGT).

SEQ ID NO: 43 is the nucleotide sequence of the oligonucleotide primer designated herein as Lib2A_R2 (CGT GGC TNW NCA CGT CCA CCA CCA CGC ATG TGA CCT CAG GGG TCC GGG AGA TCA TGA GG).

SEQ ID NO: 44 is the nucleotide sequence of the oligonucleotide primer designated herein as Lib2B_F3 (tgt gga cgt gag cnw nca cga aga ccc tga ggt caa gtt caa ctg gta cgt gga cgg cg).

SEQ ID NO: 45 is the nucleotide sequence of the oligonucleotide primer designated herein as Lib2B_R2 (CGT GNW NGC TCA CGT CCA CCA CCA CGC ATG TGA CCT CAG GGG TCC GGG AGA TCA TGA GGG).

SEQ ID NO: 46 is the nucleotide sequence of the oligonucleotide primer designated herein as lib3A-F (gtc tcc aac aaa gcc nwn ctc cca gcc ccc atc).

SEQ ID NO: 47 is the nucleotide sequence of the oligonucleotide primer designated herein as lib3A-R (GAT GGG GGC TGG GAG NWN GGC TTT GTT GGA GAC).

SEQ ID NO: 48 is the nucleotide sequence of the oligonucleotide primer designated herein as Lib3B-F (ccc aac aaa gcc ctc nwn cca gcc ccc atc gag).

SEQ ID NO: 49 is the nucleotide sequence of the oligonucleotide primer designated herein as Lib3B-R (CTC GAT GGG GGC TGG NWN GAG GGC TTT GTT GGA G).

SEQ ID NO: 50 is the nucleotide sequence of the oligonucleotide primer designated herein as Lib3C-F (cac aaa gcc ctc cca nwn gcc ccc atc gag aaa ac).

SEQ ID NO: 51 is the nucleotide sequence of the oligonucleotide primer designated herein as Lib3C-R (GTT TTC TCG ATG GGG GCN WNT GGG AGG GCT TTG TTG).

SEQ ID NO: 52 is the nucleotide sequence of the oligonucleotide primer designated herein as F1_ver1 (cag aac cac agg tgt aca ccc tgc ccc cat ccc ggg atg agc tga cca aga acc agg).

SEQ ID NO: 53 is the nucleotide sequence of the oligonucleotide primer designated herein as F2_ver1 (agc ttc tat cca agc gac atc gcc gtg cgt tgg gag agc aat ggg cag gag ctg ccg).

SEQ ID NO: 54 is the nucleotide sequence of the oligonucleotide primer designated herein as F3_ver1 (ccc cgt gct gga ctc cga cgg ctc ctt ctt cct cta cag caa gct cac cgt gga caa).

SEQ ID NO: 55 is the nucleotide sequence of the oligonucleotide primer designated herein as F4-ver1 (gct tct cct gca tgg tga tgc atg agg ctc tgc cac tcg cct tca cgc aga aga gcc).

SEQ ID NO: 56 is the nucleotide sequence of the oligonucleotide primer designated herein as R1_ver1 (tgc ttg gat aga agc ctt tga cca ggc agg tca ggc tga cct ggt tct tgg tca gct).

SEQ ID NO: 57 is the nucleotide sequence of the oligonucleotide primer designated herein as R2_ver1 (cga gtc cag cac ggg agg cgt ggt ctt gta gtt gtt ctc cgg cag ctc ctg ccc att).

SEQ ID NO: 58 is the nucleotide sequence of the oligonucleotide primer designated herein as R3_ver1 (ccc atg cag gag aag acg ttc ccc tgc tgc cac ctg ctc ttg tcc acg gtg agc ttg).

SEQ ID NO: 59 is the nucleotide sequence of the oligonucleotide primer designated herein as R4_ver1 (cgc tat aat cta gat cat tta ccc gga gac agg gag agg ctc ttc tgc gtg aag g).

SEQ ID NO: 60 is the nucleotide sequence of the oligonucleotide primer designated herein as short-F (cag aac cac agg tgt aca ccc tgc cc).

SEQ ID NO: 61 is the nucleotide sequence of the oligonucleotide primer designated herein as short-R (cct ata atc tag atc att tac c).

SEQ ID NO: 62 is the nucleotide sequence of the oligonucleotide primer designated herein as F4_ver2 (gtc ttc tcc tgc atg gtg ggc cac gag gcc ctg ccg ctg gcc ttc aca cag aag acc a).

SEQ ID NO: 63 is the nucleotide sequence of the oligonucleotide primer designated herein as R4_ver2 (cgc tat aat cta gat cat tta ccc gcc aag cgg tcg atg gtc ttc tgt gtg aag g).

SEQ ID NO: 64 is the nucleotide sequence of the oligonucleotide primer designated herein as F2_ver3 (agg ctt cta tcc aag cga cat cgc cgt tcg ctg gct gca ggg gtc aca gga gct gcc c).

SEQ ID NO: 65 is the nucleotide sequence of the oligonucleotide primer designated herein as R2_ver3 (cga gtc cag cac ggg agg cgt ggt ctt gta ctt ctc gcg ggg cag ctc ctg tga ccc).

### DETAILED DESCRIPTION OF THE INVENTION

As indicated above, the present invention provides binding proteins comprising one or more immunoglobulin constant region hinge, CH2, and/or CH3 domain(s) wherein one or more hinge and/or constant region CH2 and/or CH3 domain is modified to alter one or more of the binding protein's Fc effector function(s). Exemplified herein are binding proteins wherein the immunoglobulin hinge Fc region is modified to achieve an altered binding affinity and/or specificity for a cognate receptor (e.g., an Fc receptor) and/or to impart one or more new binding specificity(ies) to the Fc region that the corresponding unmodified binding protein does not possess (e.g., affinity for one or more Fc receptor that is distinct from the cognate receptor to which the unmodified binding protein specifically binds).

Specifically, modified binding proteins disclosed herein include the following:

(1) binding proteins comprising an insertion of one or more amino acids within an immunoglobulin hinge, CH2, and/or CH3 region, wherein the immunoglobulin exhibits an altered (i.e. an increased or decreased) binding affinity and/or specificity for one or more of FcγRI (CD64); FcγRII (CD32), including FcγRIIa, FcγRIIb, and FcγRIIc; and/or FcγRIII (CD16), including FcγRIIIa and FcγRIIIb;

(2) binding proteins comprising an insertion of one or more N-linked and/or O-linked glycosylation sequence(s) (such as, for example, one or more N-linked N-X-(S/T) glycosylation sequence(s) and/or one or more O-linked X-P-X-X (wherein at least one X is T), T-X-X-X (wherein at least one X is T), X-X-T-X (wherein at least one X is R or K), and S-X-X-X (wherein at least one X is S)) proximal to and/or distal to the site of N-linked and/or O-linked glycosylation in the corresponding native immunoglobulin Fc region, wherein the binding protein exhibits an altered (i.e. an increased or decreased) FcγR binding affinity and/or specificity; and

(3) binding proteins comprising the insertion and/or replacement of one or more amino acids within an IgG immunoglobulin CH2 and/or CH3 region wherein the amino acid insertion and/or replacement comprises one or more amino acids corresponding to an IgA immunoglobulin CH2 and/or CH3 region, wherein the one or more amino acid(s) of an IgA immunoglobulin CH2 and/or CH3 region participate in specific binding of an IgA immunoglobulin with its cognate Fcα receptor and wherein the modified binding protein is capable of specifically binding to FcαR.

Each of these embodiments of the present invention is described in further detail herein below.

The practice of the present invention will employ, unless indicated specifically to the contrary, conventional methods of immunology, molecular biology, and protein chemistry within the skill of the art, many of which are described below for the purpose of illustration. Such techniques are explained fully in the literature. See, e.g., Sambrook, et al., “Molecular Cloning: A Laboratory Manual” (2nd Edition, 1989); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); Perbal, “A Practical Guide to Molecular Cloning” (1984); Ausubel et al., “Current Protocols in Molecular Biology” (New York, John Wiley and Sons, 1987); Bonifacino et al., “Current Protocols in Cell Biology” (New York, John Wiley & Sons, 1999); Coligan et al., “Current Protocols in Immunology” (New York, John Wiley & Sons, 1999); Harlow and Lane Antibodies: a Laboratory Manual Cold Spring Harbor Laboratory (1988); and Lo, Ed., “Antibody Engineering: Methods and Protocols,” Part 1 (Humana Press, Totowa, N.J., 2004). Techniques for producing both types of mutations are well known in the art. For example, specific mutations can be introduced using site-specific mutagenesis as described in Sambrook et al., “Protocols in Molecular Biology,” supra. Random mutations in specific regions can be introduced using, for example, forced evolution as described in Gulick and Fahl, Proc. Natl. Acad. Sci. USA, 92:8140-8144 (1995).

### DEFINITIONS

As used herein, the term “binding protein” refers to proteins comprising one or more immunoglobulin heavy chain hinge, CH2, and/or CH3 domain. “Binding protein” includes and is most preferably an immunoglobulin such as an antibody or biological or functional equivalent thereof and includes parts, fragments, precursor forms, derivatives, variants, and genetically engineered forms thereof and includes labeling with chemicals and/or radioisotopes and the like. “Binding proteins” include, but are not limited to, “immunoglobulins”, “antibodies”, “monoclonal antibodies”, “chimeric antibodies”, “humanized antibodies”, and “small modular immunopharmaceutical products” (i.e. SMIP™ products) wherein one or more amino acid sequence(s) in an immunoglobulin hinge, CH2, and/or CH3 domain is altered. Within certain embodiments, the modified binding proteins disclosed herein comprise changes in one or more amino acid sequence(s) in the hinge, CH2, and/or CH3 domain that are responsible for receptor binding affinity and/or specificity.

The terms “immunoglobulin” and “antibody” broadly include all classes and subclasses of antibodies, including IgM, IgD, IgG₁, IgG₂, IgG₃, IgG₄, IgE, IgA₁ and IgA₂. The term “antibody” includes “monoclonal antibody,” which, as used herein, refers to an antibody obtained from a population of substantially homogeneous antibodies, i.e. the individual antibodies comprising the population are identical except for naturally-occurring mutations that do not substantially affect antibody binding specificity, affinity, and/or activity. “Monoclonal antibodies” include, but are not limited to, non-human monoclonal, chimeric monoclonal, humanized monoclonal, and fully-human monoclonal antibodies as well as biological or antigen-binding fragments and/or portions thereof.

As used herein, the term “chimeric antibodies” refers to monoclonal antibody molecules comprising heavy and light chains in which non-human antibody variable domains are operably fused to human constant domains. Chimeric antibodies generally exhibit reduced immunogenicity as compared to the parental fully-non-human monoclonal antibody.

As used herein, the term “humanized antibodies” refers to monoclonal antibodies comprising one or more non-human complementarity determining region (CDR), a human variable domain framework region (FR), and a human heavy chain constant domain, such as the IgG₁, IgG₂, IgG₃, and IgG₄ heavy chain constant domain and human light chain constant domain, such as the IgLambda and IgKappa light chain constant domain. As used herein, the term “humanized antibody” is meant to include human monoclonal antibodies (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, variable domain framework residues of the human antibody are replaced by corresponding non-human residues. Humanized antibodies may also comprise residues that are found neither in the recipient antibody nor in the imported CDR or framework sequences. Methods for humanizing non-human antibodies are well known in the art. Generally, a humanized antibody has one or more amino acid residue introduced into it from a source that is non-human. Humanization can be achieved by grafting CDRs into a human supporting FR prior to fusion with an appropriate human antibody constant domain. See, Jones et al., Nature 321:522-525 (1986); Riechmann et al., Nature 332:323-327 (1988); and Verhoeyen et al., Science 239:1534-1536 (1988).

As used herein, the term “fully-human antibody” refers to immunoglobulins comprising human variable regions in addition to human framework and constant regions. Such antibodies can be produced using various techniques known in the art. For example, phage display methodology have been described wherein recombinant libraries of human antibody fragments are displayed on a bacteriophage. See, McCafferty et al, Nature 348:552-554 (1990); Hoogenboom and Winter, J. Mol. Biol. 227:381 (1991); and Marks et al., J. Mol. Biol. 222:581 (1991).

Alternatively, “fully-human antibodies” may be made in transgenic animals comprising the human immunoglobulin repertoire and machinery for effecting gene rearrangement and immunoglobulin assembly. Using hybridoma technology, antigen-specific fully-human antibodies with the desired specificity may be produced and selected. One exemplary transgenic animal system is the XenoMouse® strain described by Green et al., Nature Genetics 7:13-21 (1994). The XenoMouse® strains are engineered with yeast artificial chromosomes (YACs) containing 245 kb and 190 kb germline configuration fragments, respectively, of the human heavy chain locus and kappa light chain locus that contain core variable and constant region sequences. Human Ig containing YACs are compatible with the mouse system for both rearrangement and expression of antibodies and are capable of substituting for the inactivated mouse Ig genes. More recently, Mendez et al. described the introduction of approximately 80% of the human antibody repertoire as megabase, germline configured, YAC fragments of the human heavy chain loci and kappa light chain loci. Nature Genetics 15:146-156 (1997). Transgenic animal systems suitable for generating fully-human antibodies according to the present invention have also been described in U.S. Pat. Nos. 6,150,584; 5,545,807; 5,545,806; 5,569,825; 5,625,126; 5,633,425; 5,661,016 as well as in Jakobavits, Adv Drug Deliv Rev. 31:33-42 (1998); Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg and Huszar, Intern. Rev. Immunol. 13:65-93 (1995).

In an alternative approach for generating fully-human antibodies, others have employed “miniloci” whereby an exogenous Ig locus is mimicked through the inclusion of individual gene fragments from the Ig locus. Thus, one or more V_{H} genes, one or more D_{H} genes, one or more J_{H} genes, a mu constant region, and a second constant region (typically a gamma constant region) are formed into a construct for insertion into an animal. This approach has been described in U.S. Pat. No. 5,545,807 to Surani et al. (Medical Research Council) and U.S. Pat. Nos. 5,545,806 and 5,625,825 to Lonberg and Kay (GenPharm International, Inc.) as well as in Taylor et al., International Immunology 6:579-591 (1994); Chen et al., International Immunology 5:647-656 (1993), Tuaillon et al., Proc. Natl. Acad. Sci. USA 90:3720-3724 (1993); Tuaillon et al., J. Immunol. 154:6453-6465 (1995); Choi et al. Nature Genetics 4:117-123 (1993); Lonberg et al., Nature 368:856-859 (1994); and Taylor et al., Nucleic Acids Res. 20:6287-6295 (1992).

Human antibodies avoid certain of the problems associated with antibodies that possess mouse or rat variable and/or constant regions. The presence of such mouse or rat derived sequences can lead to the rapid clearance of the antibodies or can lead to the generation of an immune response against the antibody by a patient. Thus, the use of fully-human antibodies may provide a substantial advantage in the treatment of chronic and recurring human diseases, such as inflammation, autoimmunity, and cancer, which require repeated antibody administrations.

As used herein, the term “small modular immunopharmaceutical products” (SMIP™ products) refers to a highly modular compound class having enhanced drug properties over monoclonal and recombinant antibodies. SMIP products comprise a single polypeptide chain including a target-specific binding domain, based, for example, upon an antibody variable domain, in combination with a variable FC region that permits the specific recruitment of a desired class of effector cells (such as, e.g., macrophages and natural killer (NK) cells) and/or recruitment of complement-mediated killing. Depending upon the choice of target and hinge regions, SMIP products can signal or block signalling via cell surface receptors. As used herein, engineered fusion proteins, termed “small modular immunopharmaceutical products” or “SMIP™ products”, are as described in co-owned US Patent Publication Nos. 2003/133939, 2003/0118592, and 2005/0136049, and co-owned International Patent Publications WO02/056910, WO2005/037989, and WO2005/017148, each of which is incorporated by reference herein.

Binding proteins according to the present invention are modified to alter their effector function(s) such that they bind with increased or decreased affinity and/or specificity (a) to one or more cognate Fc receptor(s) and/or (b) to one or more non-cognate Fc receptor(s). A binding protein is capable of “specifically binding” to a cognate or non-cognate receptor if it reacts at a detectable level (within, for example, an ELISA assay) with the target cognate receptor but does not react detectably with an unrelated polypeptide under similar conditions. “Specific binding,” as used in this context, generally refers to the non-covalent interactions of the type that occur between an antibody Fc region and a receptor for which the antibody Fc region is specific.

The strength, or affinity of “specific binding” interactions can be expressed in terms of the dissociation constant (K_{d}) of the interaction, wherein a smaller K_{d} represents a greater affinity. Binding properties can be quantified using methods well known in the art. One such method entails measuring the rates of target-specific binding protein/Fc receptor complex formation (i.e. association) and dissociation, wherein those rates depend upon the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al., Annual Rev. Biochem. 59:439-473 (1990). By “specifically bind” herein is meant that the binding proteins bind to target Fc receptors, proteins, and/or other molecules with a dissociation constant in the range of at least 10⁻⁶-10⁻⁹ M, more commonly at least 10⁻⁷-10⁻⁹ M.

As used in this specification and the appended claims, the singular forms “a,” “an” and “the” include plural references unless the context clearly dictates otherwise.

### Immunoglobulin Constant Region Structure

Binding proteins of the present invention comprise, in operable combination, one or more hinge, CH2, and/or CH3 domain(s) from one or more immunoglobulin selected from the group consisting of IgM, IgD, IgG₁, IgG₂, IgG₃, IgG₄, IgE, IgA₁ and IgA₂. Exemplary binding proteins comprise one or more hinge, CH2, and/or CH3 domain(s) of an IgG immunoglobulin selected from IgG₁, IgG₂, IgG₃, and IgG₄. As disclosed in detail herein, binding proteins of the present invention are altered in amino acid sequence by the insertion, deletion, and/or replacement of one or more amino acid(s) of an otherwise naturally occurring immunoglobulin hinge, CH2, and/or CH3 domain(s).

The amino acid sequences of immunoglobulin hinge, CH2, and CH3 domains are presented in the following Table 1, which is adapted from sequences provided by the International ImMunoGeneTics Information System (IMGT) which sequences are publicly available at, for example, http://imgt.cines.fr/ and disclosed herein as SEQ ID NOs 1-27.

TABLE 1

Primary Amino Acid Sequence of Human Immuno-
globulin Fc Region Hinge and CH Domains

| | | | |
|---|---|---|---|
| Immuno- | Heavy | SEQ | |
| globulin | Chain | ID | |
| Class | Domain | NO | Amino Acid Sequence |

| | | | |
|---|---|---|---|
| IgA₁ | Hinge | 1 | (V)PSTPPTPSPSTPPTPSPS |
| | CH2 | 2 | CCHPRLSLHRPALEDLLLGSEANLTCTLTGLR |
| | | | DASGVTFTWTPSSGKSAVQGPPERDLCGCYSV |
| | | | SSVLPGCAEPWNHGKTFTCTAAYPESKTPLTA |
| | | | TLSKS |
| | CH3 | 3 | (G)NTFRPEVHLLPPPSEELALNELVTLTCLA |
| | | | RGFSPKDVLVRWLQGSQELPREKYLTWASRQE |
| | | | PSQGTTTFAVTSILRVAAEDWKKGDTFSCMVG |
| | | | HEALPLAFTQKTIDRLAGKPTHVNVSVVMAEV |
| | | | DGTCY |
| IgA₂ | Hinge | 4 | (V)PPPPP |
| | CH2 | 5 | CCHPRLSLHRPALEDLLLGSEANLTCTLTGLR |
| | | | DASGATFTWTPSSGKSAVQGPPERDLCGCYSV |
| | | | SSVLPGCAQPWNHGETFTCTAAHPELKTPLTA |
| | | | NITKS |
| | CH3 | 6 | (G)NTFRPEVHLLPPPSEELALNELVTLTCLA |
| | | | RGFSPKDVLVRWLQGSQELPREKYLTWASRQE |
| | | | PSQGTTTFAVTSILRVAAEDWKKGDTFSCMVG |
| | | | HEALPLAFTQKTIDRLAGKPTHVNVSVVMAEV |
| | | | DGTCY |
| IgD | Hinge | 7 | (E)SPKAQASSVPTAQPQAEGSLAKATTAPAT |
| | | | TRNT(G)RGGEEKKKEKEKEEQEERETKTP |
| | CH2 | 8 | (E)CPSHTQPLGVYLLTPAVQDLWLRDKATFT |
| | | | CFVVGSDLKDAHLTWEVAGKVPTGGVEEGLLE |
| | | | RHSNGSQSQHSRLTLPRSLWNAGTSVTCTLNH |
| | | | PSLPPQRLMALREP |
| | CH3 | 9 | (A)AQAPVKLSLNLLASSDPPEAASWLLCEVS |
| | | | GFSPPNILLMWLEDQREVNTSGFAPARPPPQP |
| | | | RSTTFWAWSVLRVPAPPSPQPATYTCVVSHED |
| | | | SRTLLNASRSLEVS(Y)VTDHGPMK |
| IgE | CH2 | 10 | (V)CSRDFTPPTVKILQSSCDGGGHFPPTIQL |
| | | | LCLVSGYTPGTINITWLEDGQVMDVDLSTAST |
| | | | TQEGELASTQSELTLSQKHWLSDRTYTCQVTY |
| | | | QGHTFEDSTKKCA |
| | CH3 | 11 | (D)SNPRGVSAYLSRPSPFDLFIRKSPTITCL |
| | | | VVDLAPSKGTVNLTWSRASGKPVNHSTRKEEK |
| | | | QRNGTLTVTSTLPVGTRDWIEGETYQCRVTHP |
| | | | HLPRALMRSTTKTS |
| | CH4 | 12 | (G)PRAAPEVYAFATPEWPGSRDKRTLACLIQ |
| | | | NFMPEDISVQWLHNEVQLPDARHSTTQPRKTK |
| | | | GSGFFVFSRLEVTRAEWEQKDEFICRAVHEAA |
| | | | SPSQTVQRAVSVNPGK |
| IgG₁ | Hinge | 13 | (E)PKSCDKTHTCPPCP |
| | CH2 | 14 | (A)PELLGGPSVFLFPPKPKDTLMISRTPEVT |
| | | | CVVVDVSHEDPEVKFNWYVDGVEVHNAKTKPR |
| | | | EEQYNSTYRVVSVLTVLHQDWLNGKEYKCKVS |
| | | | NKALPAPIEKTISKAK |
| | CH3 | 15 | (G)QPREPQVYTLPPSRDELTKNQVSLTCLVK |
| | | | GFYPSDIAVEWESNGQPENNYKTTPPVLDSDG |
| | | | SFFLYSKLTVDKSRWQQGNVFSCSVMHEALHN |
| | | | HYTQKSLSLSPGK |
| IgG₂ | Hinge | 16 | (E)RKCCVECPPCP |
| | CH2 | 17 | (A)PPVAGPSVFLFPPKPKDTLMISRTPEVTC |
| | | | VVVDVSHEDPEVQFNWYVDGVEVHNAKTKPRE |
| | | | EQFNSTFRVVSVLTVVHQDWLNGKEYKCKVSN |
| | | | KGLPAPIEKTISKTK |
| | CH3 | 18 | (G)QPREPQVYTLPPSREEMTKNQVSLTCLVK |
| | | | GFYPSDIAVEWESNGQPENNYKTTPPMLDSDG |
| | | | SFFLYSKLTVDKSRWQQGNVFSCSVMHEALHN |
| | | | HYTQKSLSLSPGK |
| IgG₃ | Hinge | 19 | (E)LKTPLGDTTHTCPRCP(E)PKSCDTPPPC |
| | | | PRCP(E)PKSCDTPPPCPRCP(E)PKSCDTPP |
| | | | PCPRCP |
| | CH2 | 20 | (A)PELLGGPSVFLFPPKPKDTLMISRTPEVT |
| | | | CVVVDVSHEDPEVQFKWYVDGVEVHNAKTKPR |
| | | | EEQYNSTFRVVSVLTVLHQDWLNGKEYKCKVS |
| | | | NKALPAPIEKTISKTK |
| | CH3 | 21 | (G)QPREPQVYTLPPSREEMTKNQVSLTCLVK |
| | | | GFYPSDIAVEWESSGQPENNYNTTPPMLDSDG |
| | | | SFFLYSKLTVDKSRWQQGNIFSCSVMHEALHN |
| | | | RFTQKSLSLSPGK |
| IgG₄ | Hinge | 22 | (E)SKYGPPCPSCP |
| | CH2 | 23 | (A)PEFLGGPSVFLFPPKPKDTLMISRTPEVT |
| | | | CVVVDVSQEDPEVQFNWYVDGVEVHNAKTKPR |
| | | | EEQFNSTYRVVSVLTVLHQDWLNGKEYKCKVS |
| | | | NKGLPSSIEKTISKAK |
| | CH3 | 24 | (G)QPREPQVYTLPPSQEEMTKNQVSLTCLVK |
| | | | GFYPSDIAVEWESNGQPENNYKTTPPVLDSDG |
| | | | SFFLYSRLTVDKSRWQEGNVFSCSVMHEALHN |
| | | | HYTQKSLSLSLGK |
| IgM | CH2 | 25 | (V)IAELPPKVSVFVPPRDGFFGNPRKSKLIC |
| | | | QATGFSPRQIQVSWLREGKQVGSGVTTDQVQA |
| | | | EAKESGPTTYKVTSTLTIKESDWLGQSMFTCR |
| | | | VDHRGLTFQQNASSMCVP |
| | CH3 | 26 | (D)QDTAIRVFAIPPSFASIFLTKSTKLTCLV |
| | | | TDLTTYDSVTISWTRQNGEAVKTHTNISESHP |
| | | | NATFSAVGEASICEDDWNSGERFTCTVTHTDL |
| | | | PSPLKQTISRPK |
| | CH4 | 27 | (G)VALHRPDVYLLPPAREQLNLRESATITCL |
| | | | VTGFSPADVFVQWMQRGQPLSPEKYVTSAPMP |
| | | | EPQAPGRYFAHSILTVSEEEWNTGETYTCVVA |
| | | | HEALPNRVTERTVDKSTGKPTLYNVSLVMSDT |
| | | | AGTCY |

Immunoglobulin hinge region polypeptides occur naturally in immunoglobulins of the IgG, IgA, and IgD classes. A major structural difference between IgG₁, IgG₂, IgG₃, and IgG₄ is the length of the hinge region. In immunoglobulin heavy chain, wild-type immunoglobulin hinge region polypeptides are situated between CH1 and CH2 regions and contain cysteine residues that are responsible for forming intrachain disulfide bonds.

As is known to the art, despite the tremendous overall diversity in immunoglobulin amino acid sequences, immunoglobulin primary structure exhibits a high degree of sequence conservation in particular portions of immunoglobulin polypeptide chains, notably with regard to the occurrence of cysteine residues which, by virtue of their sulfhydryl groups, offer the potential for disulfide bond formation with other available sulfydryl groups. Accordingly, in the context of the present invention wild-type immunoglobulin hinge region polypeptides include those that feature one or more highly conserved cysteine residues. The wild-type human IgG₁ hinge region polypeptide sequence comprises three non-adjacent cysteine residues, referred to as a first cysteine of the wild-type hinge region, a second cysteine of the wild-type hinge region and a third cysteine of the wild-type hinge region, respectively, proceeding along the hinge region sequence from the polypeptide N-terminus toward the C-terminus.

Immunoglobulin IgA, IgD, and IgG Fc regions comprise a single CH2 and a single CH3 domain whereas IgE and IgM Fc regions comprise a single CH2, a single CH3 domain, and a single CH4 domain. While the percent identity between the four subclasses of IgG Fc regions (i.e. IgG₁, IgG₂, IgG₃, and IgG₄) is in excess of 95%, these regions possess dramatically different FcγR binding specificities (see, Table 2, below).

TABLE 2
Relative Human Fcγ Receptor Recognition Specificity
between IgG Immunoglobulin Subclasses

| | IgG₁ | IgG₂ | IgG₃ | IgG₄ |
|---|---|---|---|---|
| FcγRI | +++ | − | +++ | ++ |
| FcγRII | + | − | + | − |
| FcγRIII | + | − | + | − |

Within certain embodiments, binding proteins of the present invention are capable of antibody-dependent cellular cytotoxicity (ADCC), complement-dependent cellular cytotoxicity (CDC), and/or complement fixation. The present invention offers unexpected advantages associated with retention by the binding proteins described herein of the ability to mediate ADCC and/or CDC and/or complement fixation notwithstanding any alteration in the binding protein's binding affinity and/or specificity for one or more cognate and/or non-cognate receptor. Manipulation of sequences encoding antibody constant region domains is referenced in Morrison and Oi, U.S. Pat. No. 6,218,149.

### Amino Acid Insertion Mutations that Alter IgG-Based Immunoglobulin Fcγ-Receptor Binding Affinity and/or Specificity

In one aspect, the present invention provides binding proteins, in particular binding proteins comprising one or more immunoglobulin heavy chain hinge, CH2, and/or CH3 domain, wherein the binding protein is modified such that it binds with altered (i.e. either increased or decreased) binding affinity and/or specificity to one or more immunoglobulin-specific Fc receptor. Binding proteins provided herein also include those wherein one or more amino acid residue(s) is inserted into one or more amino acid sequences in the constant region. In one aspect, one or more amino acid residues is inserted into one or more amino acid sequence(s) that makes direct contact with a receptor, one or more amino acid sequences that are adjacent to an amino acid sequence that makes direct contact with a receptor, one or more amino acid sequence(s) distal from an amino acid sequence that makes direct contact with a receptor, or various combinations of these sequences. Inserted amino acid residues can introduce a localized or overall conformational change in the immunoglobulin three-dimensional structure that alters binding affinity and/or specificity to a cognate receptor.

Within certain embodiments, the inserted amino acid residues comprise an amino acid sequence that is identical to an existing amino acid sequence in the binding protein's hinge, CH2, and/or CH3 domain that makes direct contact with a cognate receptor upon binding. In one such embodiment, one or more amino acid sequence that makes direct contact with the receptor are positioned in tandem with respect to the position of the “wild-type” receptor binding amino acid sequence. “Wild-type” as used in this context refers to the amino acid sequence of the binding protein into which changes are to be introduced or the nucleotide sequence of the polynucleotide encoding the binding protein.

In another aspect, the binding protein includes one or more inserted receptor binding sequence(s) identical to a wild-type receptor binding sequence that is introduced into the constant region of the binding protein at a site that is distal from and on the same chain as a wild-type receptor binding sequence and/or at a site on a chain in the binding protein on which the wild-type receptor binding sequence is not located, or both. Regardless of the precise nature and sequence of the amino acid insertion, modifications of this type are well know and routinely practiced in the art as described in Sambrook et al., “Protocols in Molecular Biology,” supra.

Exemplified herein are binding proteins, in particular binding proteins comprising one or more IgG heavy chain hinge, CH2, and/or CH3 domain, wherein the binding protein is modified such that it binds with altered (i.e. either increased or decreased) binding affinity and/or specificity to one or more immunoglobulin-specific Fc receptor including, but not limited to, one or more of the IgG immunoglobulin-specific receptors FcγRI (CD64), FcγRII (CD32), and/or FcγRIII (CD16). Binding proteins of this type include, for example, binding proteins wherein one or more amino acid(s) is inserted within the binding protein's primary amino acid sequence, within the hinge, CH2, and/or CH3 domain, in sequences that are responsible for Fcγ-receptor binding. Such changes include, but are not limited to, the insertion of one or more amino acid(s) between and/or adjacent to amino acids that contribute by direct contact to the association of the binding protein with one or more immunoglobulin-specific Fc receptor(s) including FcγRI (CD64), FcγRII (CD32), and/or FcγRIII (CD16).

Specific embodiments of these aspects of the present invention include binding proteins comprising one or more IgG CH2 domain wherein the CH2 domain is an IgG₁ and/or an IgG₃ CH2 domain. Some such embodiments provide binding proteins comprising one or more amino acid deletion from and/or amino acid insertion within the hinge proximal loop structure, L-L-G-G-P, of the IgG₁ and/or IgG₃ CH2 domain. Specifically exemplified herein are binding proteins comprising single insertions of a single amino acid at the positions indicated by the “*” within the following hinge proximal loop structure. Thus, provided herein are binding proteins comprising the modified hinge proximal loop structures L-L-*-G-G-P, L-L-G-*-G-P, and L-L-G-G-*-P. Also provided herein are binding proteins comprising single insertions of two or more amino acids at the positions indicated by “*” within the hinge proximal loop structures L-L-*-G-G-P, L-L-G-*-G-P, and L-L-G-G-*-P. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified hinge proximal loop structures are selected from the group consisting of Ala, Gly, Ile, Leu, and Val.

Other such embodiments provide binding proteins comprising one or more IgG CH2 domain wherein the CH2 domain is an IgG₁, IgG₂, and/or IgG₃ CH2 domain. Some such embodiments provide binding proteins comprising one or more amino acid deletion from and/or amino acid insertion within the BC loop structure, D-V-S-H-E, of the IgG₁, IgG₂, and/or IgG₃ CH2 domain. Specifically exemplified herein are binding proteins comprising single insertions of a single amino acid at the positions indicated by the “*” within the following BC loop structure. Thus, provided herein are binding proteins comprising the modified BC loop structures D-V-*-S-H-E and D-V-S-*-H-E. Also provided herein are binding proteins comprising single insertions of two or more amino acids at the positions indicated by “*” within the BC loop structures D-V-*-S-H-E and D-V-S-*-H-E. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified BC loop structures are selected from the group consisting of Ala, Gly, Ile, Leu, and Val.

Still other such embodiments provide binding proteins comprising one or more IgG CH2 domain wherein the CH2 domain is an IgG₁ and/or IgG₃ CH2 domain. Some such embodiments provide binding proteins comprising one or more amino acid deletion from and/or amino acid insertion within the FG loop structure, A-L-P-A-P-I, of the CH2 domain. Specifically exemplified herein are binding proteins comprising single insertions of a single amino acid at the positions indicated by the “*” within the following FG loop structure. Thus, provided herein are binding proteins comprising the modified FG loop structures A-L-*-P-A-P-I, A-L-P-*-A-P-I, and A-L-P-A-*-P-I. Also provided herein are binding proteins comprising single insertions of two or more amino acids at the positions indicated by “*” within the FG loop structures A-L-*-P-A-P-I, A-L-P-*-A-P-I, and A-L-P-A-*-P-I. Thus, within these embodiments, “*” indicates the insertion of at least 2, 3, 4, 5, 6, 7, 8, 9, 10, 11, 12, 13, 14, 15, 16, 17, 18, 19, or 20 amino acids. Typically, amino acids suitable for the generation of binding proteins having such modified FG loop structures are selected from the group consisting of Ala, Gly, Ile, Leu, and Val.

The spacing, in terms of the number of intervening amino acid residues, between a first inserted receptor binding sequence and a second inserted receptor binding sequence and between a second inserted receptor binding sequence and a third inserted receptor binding sequence and so on, can range from between zero (0) intervening amino acid residues and about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid residues. The exact number of intervening amino acid residues, if present, between a first inserted receptor binding sequence and a second inserted receptor binding sequence and between a second inserted receptor binding sequence and a third inserted receptor binding sequence will vary in a manner so as to increase receptor binding affinity. In modified binding proteins comprising more than two receptor binding sites, the number of intervening amino acid residues between receptor binding sequences can be the same or can be different. Intervening amino acid residues in the spacer regions may be specifically selected or may be randomly selected by assaying binding affinity.

Within one exemplary embodiment, binding protein functionality, for example G-SMIP™-product functionality, may be altered, in the manner described herein above, by changing the binding of a modified G-SMIP™-product to increase or decrease FcγRI (CD64) binding, FcγRII (CD32) binding, and/or FcγRIIIa (CD16) binding. Within certain aspects of these exemplary embodiments, such changes to immunoglobulin functionality are effective in increasing or decreasing, respectively, the corresponding antibody-dependent cellular cytotoxicity (ADCC). Within certain such embodiments, the binding protein is a G-SMIP product wherein functionality is altered, as described above, by inserting and/or deleting amino acid sequences within Fcγ loops. Within related embodiments, the binding protein is G-SMIP product wherein functionality is altered by changing loop contacts with FcγR and/or C1q. For example, amino acids may be inserted and/or deleted to alter binding face for FcγRs or to lower CDC by decreasing C1q binding.

Inserting amino acid residues into the binding protein's amino acid sequence can be directed, for example, through one or more insertion mutation(s) within a polynucleotide encoding the immunoglobulin amino acid sequence, or through random mutations in specific regions as described herein.

FcγRI binding, FcγRII binding, FcγRIII binding, and FcαRIII binding may be assessed by measuring, respectively, binding to CD64, CD32, CD16, and CD89 by methodology well known in the art and as described in further detail herein below.

### Mutations Comprising Glycosylation Sequences that Alter IgG-Based Immunoglobulin Fcγ-Receptor Binding Affinity and/or Specificity

Binding proteins of the invention, as described herein may, according to certain embodiments, desirably comprise additional sites for glycosylation, e.g., covalent attachment of carbohydrate moieties such as, for example, monosaccharides or oligosaccharides. Incorporation of amino acid sequences that provide substrates for polypeptide glycosylation is within the scope of the relevant art, including, for example, the use of genetic engineering or protein engineering methodologies to obtain a polypeptide sequence containing, for example, the classic Asn-X-Ser/Thr site for N-(asparagine)-linked glycosylation, or a sequence containing Ser or Thr residues that are suitable substrates for O-linked glycosylation, or sequences amenable to C-mannosylation, glypiation/glycosylphosphatidylinositoI modification, or phosphoglycation, all of which can be identified according to art-established criteria (e.g., Spiro, Glybiology 12:43 R (2002)).

It is believed that N-linked glycosylation of the immunoglobulin CH2 DE loop alters the CH2 conformation and provides direct sugar contacts with FcγR. Modifications within immunoglobulin glycoforms may be achieved by inserting one or more amino acid sequence comprising an N-linked glycosylation site such as, for example, the amino acid sequence YNSTY. Such new glycoforms may have altered FCγR binding properties. Within an alternative aspect of such embodiments, a second N-linked glycosylation site may be inserted within a CH2 DE loop. For example, the wild-type sequence YNSTY may be converted to YNSTYNSTY. Such immunoglobulin modifications will substantially affect FcγR binding and, consequently, antibody dependent cellular cytotoxicity (ADCC).

It has been shown that N-linked glycosylation, in particular N-linked glycosylation at amino acid position Asn297, is essential for binding of IgG class immunoglobulins to their cognate Fcγ receptor. Thus, for example, mutation of Asn297 to Ala297 has been shown to abrogate recognition of FcγRI. It has similarly been shown that, in the absence of the N-linked oligosaccharide at position Asn297, recognition and/or activation of FcγRI, FcγRII, FcγRIII (as well as complement C1q) is abrogated whereas Protein A and rheumatoid factor binding are unaffected. N- and O-linked glycosylation sequences are well known in the art as described, for example, in Gooley et al., Biochem. Biophys. Res. Commun. 178:1194-1201 (1991) and Pisano et al., Glycobiology 3:429-435 (1993).

Within certain aspects, the present invention provides modifications that include insertion of one or more amino acid(s) into and/or deletion of one or more amino acid(s) from one or more hinge region and/or constant region immunoglobulin loop(s) comprising one or more O- and/or N-linked glycosylation site(s).

Accordingly, the present invention provides binding proteins, in particular binding proteins comprising one or more heavy chain hinge, CH2, and/or CH3 domain, wherein the binding protein comprises one or more modification(s) within the one or more heavy chain hinge, CH2, and/or CH3 domain wherein the modification comprises the insertion of one or more N-linked glycosylation sequence(s) and/or one or more O-linked glycosylation sequence(s), which glycosylation sequence is sufficient to achieve N- and/or O-linked glycosylation at the position of insertion thereby altering (i.e. either increasing or decreasing) the binding protein's binding affinity and/or specificity to one or more immunoglobulin-specific Fc receptor or other target protein. Binding proteins of this type include, for example, those comprising changes in the primary amino sequence at positions that are proximal and/or distal to regions, domains, and/or loop structures responsible for glycosylation in the unmodified binding protein.

Exemplified herein are binding proteins comprising one or more IgG heavy chain hinge, CH2, and/or CH3 domain, wherein the binding proteins comprise one or more modification(s) within the one or more IgG heavy chain hinge, CH2, and/or CH3 domain wherein the modification comprises the insertion of one or more N-linked glycosylation sequence(s) and/or one or more O-linked glycosylation sequence(s), which glycosylation sequence is sufficient to achieve N- and/or O-linked glycosylation at the position of insertion thereby altering (i.e. either increasing or decreasing) the binding protein's binding affinity and/or specificity to one or more IgG immunoglobulin-specific receptor(s) FcγRI (CD64), FcγRII (CD32), and/or FcγRIII (CD16) as compared to a corresponding binding protein comprising one or more unmodified IgG heavy chain hinge, CH2, and/or CH3 domain.

Specific embodiments of these aspects of the present invention include binding proteins comprising one or more IgG hinge domain, one or more IgG CH2 domain, and/or one or more IgG CH3 domain wherein the hinge, CH2, and/or CH3 domain is an IgG₁ hinge, CH2, and/or CH3 domain, an IgG₂ hinge, CH2, and/or CH3 domain, an IgG₃ hinge, CH2, and/or CH3 domain, and/or an IgG₄ hinge, CH2, and/or CH3 domain. Some such embodiments provide binding proteins comprising the insertion of one or more N-linked glycosylation sequence N-X-(S/T) (wherein X is any amino acid) and/or one or more O-linked glycosylation sequence X-P-X-X (wherein at least one X is T), T-X-X-X (wherein at least one X is T), X-X-T-X (wherein at least one X is R or K), and/or S-X-X-X (wherein at least one X is S)) proximal to and/or distal to the site of N-linked and/or O-linked glycosylation in the corresponding native IgG immunoglobulin IgG₁ hinge, CH2, and/or CH3 domain. Within certain aspects of these embodiments, the binding protein exhibits an altered (i.e. an increased or decreased) FcγR binding affinity and/or specificity.

Exemplified herein are such embodiments wherein the binding proteins comprise one or more IgG hinge domain, one or more IgG CH2 domain, and/or one or more IgG CH3 domain and wherein the binding proteins further comprise an insertion of one or more N-linked glycosylation sequence N-X-(S/T) (wherein X is any amino acid). For example, the present invention provides such binding proteins comprising an insertion of one or more N-X-(S/T) sequence adjacent to the native N-S-T sequence within the DE loop of one or more IgG₁, IgG₂, IgG₃, and/or IgG₄ CH2 domain. Within certain aspects of these embodiments, the binding protein exhibits an altered (i.e. an increased or decreased) FcγR binding affinity and/or specificity.

Within specific such embodiments, the N-linked glycosylation sequence within the DE loop of one or more IgG₁, IgG₂, IgG₃, and/or IgG₄ CH2 domain comprises the amino acid sequence N-S-T and is inserted adjacent to and/or within 0 to 100 amino acids amino-terminal and/or carboxy-terminal to the native N-S-T sequence such that the native amino acid sequence X-N-S-T-Z is modified to (AA^{a})-N-S-T-(AA^{b})-N-S-T-(AA^{c}) wherein each of AA^{a}, AA^{b}, and AA^{c} independently designate from 1 to 100 amino acids.

Within specific such embodiments, the N-linked glycosylation sequence within the DE loop of one or more IgG₁, IgG₂, IgG₃, and/or IgG₄ CH2 domain comprises the amino acid sequence N-S-T and is inserted adjacent to the native N-S-T sequence such that the native amino acid sequence X-N-S-T-Z is modified to X-N-S-T-Z-N-S-T-Z, wherein X and Z are independently selected from Tyr (Y) and Phe (F).

Within alternative such embodiments, the N-linked glycosylation sequence inserted within the BC loop of one or more IgG₁, IgG₂, IgG₃, and/or IgG₄ CH3 domain comprises the amino acid sequence N-S-T and is inserted distal to the native N-S-T sequence such that the native amino acid sequence Y-P-S-D-I-A is modified to Y-P-N-S-T-D-I-A and Y-N-S-T-P-S-D-I-A.

In another aspect, the present invention provides binding proteins, in particular binding proteins comprising one or more IgG heavy chain hinge, CH2, and/or CH3 domain, wherein the binding protein comprises one or more modification(s) within the one or more IgG heavy chain hinge, CH2, and/or CH3 domain wherein the modification comprises the insertion of one or more N-linked glycosylation sequence(s) and/or one or more O-linked glycosylation sequence(s), which glycosylation sequence is sufficient to achieve N- and/or O-linked glycosylation at the position of insertion thereby altering (i.e. either increasing or decreasing) the binding protein's binding affinity and/or specificity to one or more immunoglobulin-specific Fc receptor including the IgG immunoglobulin-specific receptors FcγRI (CD64), FcγRII (CD32), and/or FcγRIII (CD 16) as compared to a corresponding binding protein comprising one or more unmodified IgG heavy chain hinge, CH2, and/or CH3 domain. Binding proteins of this type include, for example, those comprising changes in the primary amino sequence at positions that are proximal and/or distal to regions, domains, and/or loop structures responsible for glycosylation in the unmodified binding protein.

Such changes include, for example, the insertion of three or more amino acids comprising the YNS sequence for N-linked glycosylation between and/or adjacent to amino acids that, upon binding, are in contact with one or more immunoglobulin-specific Fc receptor including FcγRI (CD64), FcγRII (CD32), and/or FcγRIII (CD16).

One such aspect of the presently described embodiment provides modifications within the DE loop of the CH2 domain of the IgG class of immunoglobulins referred to as G-SMIP™-products, which contain one or more amino acid sequence YNSTY that is a site for N-linked glycosylation and for FcγR contacts.

### Binding Proteins Having Both Cognate Receptor and Non-Cognate Receptor Binding Specificities

Within still further aspects, the present invention provides binding proteins, wherein a new functionality is achieved by replacing one or more immunoglobulin loop(s) of a first immunoglobulin class with one or more second immunoglobulin loop(s) of a second immunoglobulin class wherein the second immunoglobulin loop(s) imparts a new binding specificity to the modified binding protein that is not present in the corresponding unmodified binding protein.

Binding proteins according to these aspects of the present invention comprise one or more heavy chain hinge, CH2, and/or CH3 domain of a first immunoglobulin class (i.e. IgA, IgD, IgE, IgG, or IgM), wherein the binding protein is modified (i.e. by amino acid replacement and/or amino acid insertion) in the primary amino sequence of one or more heavy chain hinge, CH2, and/or CH3 domain of the first immunoglobulin class to generate a binding protein capable of binding to one or more cognate Fc receptor of a second immunoglobulin class distinct from the first immunoglobulin class. Such changes include, for example, replacing and/or remodeling one or more loops, or amino acid and/or peptide portions thereof, of a first immunoglobulin domain with one or more loops, or amino acid and/or peptide portions thereof, of a second immunoglobulin domain, wherein the second immunoglobulin domain comprises one or more amino acids that form at least a portion of a binding sequence for a second immunoglobulin-specific Fc receptor. Binding proteins according to these aspects of the present invention are capable of specifically binding to FcαR in addition to being capable of specifically binding to FcγRI, FcγRII, and/or FcγRIII.

Exemplified herein are binding proteins comprising one or more IgG heavy chain hinge, CH2, and/or CH3 domain, wherein the binding protein is modified to bind to one or more non-IgG immunoglobulin-specific Fc receptor including, but not limited to, the IgA immunoglobulin-specific receptor FcαR (CD89). Binding proteins of this type include, for example, binding proteins comprising changes (i.e. amino acid replacement and/or amino acid insertion) in the primary amino sequence of one or more IgG heavy chain hinge, CH2, and/or CH3 domain to generate amino acid sequences capable of non-IgG immunoglobulin-specific Fc receptor binding such as, for example, Fcα-receptor binding.

Within certain such embodiments are provided binding proteins comprising one or more IgG heavy chain hinge, CH2, and/or CH3 domain, wherein the binding protein is modified to bind to the IgA immunoglobulin-specific receptor FcαR (CD89). Such exemplary binding proteins comprise one or more amino acid substitution(s) within the IgG CH3 FG loop and/or one or more amino acid substitution(s) within the IgG CH3 CD loop.

For example, one such exemplary binding protein comprises the replacement of the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof. Another exemplary binding protein comprises the replacement of the IgG CH3 CD loop comprising the amino acid sequence Q-P-E-N, or a portion thereof, with the IgA CH3 CD loop comprising the amino acid sequence Q-E-L-P-R-E, or a portion thereof.

Yet another such exemplary binding protein comprises the replacement of both the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof, and the IgG CH3 CD loop comprising the amino acid sequence Q-P-E-N, or a portion thereof, with the IgA CH3 CD loop comprising the amino acid sequence Q-E-L-P-R-E, or a portion thereof.

Any of the aforementioned binding protein embodiments may further comprise the substitution of IgG heavy chain CH3 amino acid Met (at CH3 amino acid position no. 28 within the sequence K-D-T-L-M₂₈-I-S-R-T) with amino acid Leu such that the binding protein further comprises the amino acid sequence K-D-T-L-L₂₈-I-S-R-T. Alternatively or additionally, any of the aforementioned binding protein embodiments may further comprise the substitution of IgG heavy chain CH3 amino acid Glu (at CH3 amino acid position no. 157 within the sequence D-I-A-V-E₁₅₇-W-E-S-N) with amino acid Arg such that the binding protein further comprises the amino acid sequence D-I-A-V-R₁₅₇-W-E-S-N.

Exemplified herein, within certain embodiments, a loop comprising a binding contact for a non-FcγR is inserted into the IgG-based binding protein comprising one or more IgG heavy chain hinge, CH2, and/or CH3 domain, wherein the binding proteins are modified to bind to one or more non-IgG immunoglobulin-specific Fc receptor including, but not limited to, the IgA immunoglobulin-specific Fc receptor FcαR (CD89). Binding proteins of this type include, for example, binding proteins comprising one or more change(s) (i.e. amino acid replacement and/or amino acid insertion) in the primary amino sequence of one or more IgG heavy chain hinge, CH2, and/or CH3 domain to generate amino acid sequences capable of non-IgG immunoglobulin-specific Fc receptor binding such as, for example, Fcα-receptor binding. Such changes include, for example, replacing and/or remodeling one or more IgG immunoglobulin loops with one or more non-IgG immunoglobulin loop(s) and/or peptide portions thereof, wherein the non-IgG immunoglobulin loop comprises a binding sequence for a non-IgG immunoglobulin-specific Fc receptor.

Within certain embodiments, the binding protein is a G-SMIP product wherein G-SMIP product functionality is altered such that the G-SMIP product binds to one or more FcαR such as CD89. Several loops on the CH3 domain of IgG are engineered, as described above, to provide new and/or modified molecular interactions. For example, IgG hinge, CH2, and/or CH3 amino acids may be replaced with IgA hinge, CH2, and/or CH3 residues that participate in binding between IgA and FcαR. For example, and as described above for binding proteins generally, the IgG CH3 FG loop may be replaced with an IgA CH3 FG loop plus other amino acids that contact FcαR. Alternatively or additionally, the IgG CH3 CD loop may be replaced with the IgA CH3 CD loop plus other amino acids that contact the FcαR. Exemplary G-SMIP products comprise an amino-terminal end from the humanized antibody designated 2H7-018014. G-SMIP™ products comprising, for example, amino acids that confer FcαR binding activity retain the benefits of the unmodified G-based SMIP™ products such as, for example, long in vivo half-life, ease of purification by protein A, and/or IgG effector functions.

Still further embodiments provide modifications in the binding protein's specificity for non-antibody receptor binding such as, for example, binding to T cell surface proteins; B cell surface proteins; myeloid cell surface proteins; and non-immune cell proteins.

### Methodology for Generating, Expressing, and Characterizing Functionality of Binding Proteins Having Altered Effector Function

Once a binding protein, as provided herein, has been designed, polynucleotides including DNAs encoding the binding protein may be synthesized in whole or in part via oligonucleotide synthesis as described, for example, in Sinha et al., Nucleic Acids Res., 12:4539-4557 (1984); assembled via PCR as described, for example in Innis, Ed. “PCR Protocols” (Academic Press, 1990) and also in Better et al., J. Biol. Chem. 267:16712-16118 (1992). Methodology for sequencing, cloning, and expressing such unmodified and modified binding proteins are known in the art by reference, for example, to procedures as described in Ausubel et al., Eds. “Current Protocols in Molecular Biology” (John Wiley & Sons, New York, 1989) and also in Robinson et al., Hum. Antibod. Hybridomas 2:84-93 (1991). Binding proteins of the present invention may be expressed in a eukaryotic cell line (such as, for example, a CHO cell line), purified via Protein A chromatography, and characterized by functional assays.

Expression may be achieved in any conventional mammalian expression system known in the art by isolating a DNA fragment encoding the binding protein of interest and cloning into a mammalian expression vector such as, for example, pD18. DNA from positive clones may be amplified using QIAGEN plasmid preparation kits (QIAGEN, Valencia, Calif.). The recombinant plasmid DNA may be linearized in a nonessential region by digestion with a suitable restriction endonuclease, purified by phenol extraction, and resuspended in tissue culture media (e.g., Excell 302; Catalog #14312-79P, JRH Biosciences, Lenexa, Kans.). Cells suitable for transfection are, for example, CHO DG44 cells, typically in a logarithmic growth stage. Cells are harvested for each transfection reaction and linearized DNA is added to the cells for transfection or electroporation. For example, stable production of inventive binding proteins may be achieved by electroporation of CHO cells with a selectable, amplifiable plasmid, such as pD18, containing the cDNA encoding the binding protein under the control of the CMV promoter. (All cell lines are available from the American Type Culture Collection; Manassas, Va.). An expression cassette comprising the binding protein cDNA may be subcloned downstream of a suitable promoter (such as the CMV promoter).

Transfected cells are allowed to recover overnight in non-selective media prior to selective plating in a 96-well flat bottom plate (Costar) at varying serial dilutions ranging from, for example, 125 cells/well to 2000 cells/well with suitable culture media for cell cloning such as Excell 302 complete medium, containing selective agent (such as, for example, 100 nM methotrexate in the case of DHFR resistance). Serial dilutions of culture supernatants from master wells are screened for binding to cells expressing the relevant binding protein ligand.

Supernatants are typically collected from CHO cells expressing the binding protein, filtered through 0.2 μm filters (Nalgene, Rochester, N.Y.), and passed over a Protein A-agarose (IPA 300 crosslinked agarose) column (Repligen, Needham, Mass.). The column is washed with PBS and bound protein is eluted using 0.1 M citrate buffer, pH 3. Fractions are collected and eluted protein neutralized using 1M Tris, pH 8.0, prior to dialysis into PBS. The concentration of purified binding protein may be determined by absorption at 280 nm.

Binding proteins may be tested for desired activity, for example, binding to a target receptor such as FcγRI, FcγRII, FcγRIII and/or FcαR, or specific antigen binding activity, as described, for example, in Harlow et al., Eds. “Antibodies: A Laboratory Manual” Chapter 14 (Cold Spring Harbor Laboratory, Cold Spring Harbor, 1988) and Munson et al., Anal. Biochem. 107:220-239 (1980) as well as antibody dependent cell-mediated cytotoxicity (ADCC) and complement dependent cytotoxicity (CDC) by methods known in the art. ADCC and CDC assays, secondary in vitro antibody responses, flow immunocytofluorimetric analyses of various peripheral blood or lymphoid mononuclear cell subpopulations using well established marker antigen systems, immunohistochemistry, and other relevant assays are, for example, all provided herein by reference to Rose et al. Eds. “Manual of Clinical Laboratory Immunology” (American Society of Microbiology, Washington, D.C., 1997).

The ability of binding proteins to mediate ADCC may be measured using any suitable target cell line and PBMCs as the effector cells. For example, in the specific case of CD20-specific binding proteins, suitable cell lines include the B-cell lines Ramos and Bjab. Effector to target ratios are typically varied, for example, as follows: 100:1, 50:1, 25:1, and 10:1, with the number of target cells per well remaining constant while varying the number of PBMCs. Target cells are labeled with ⁵¹Cr (e.g., Na₂⁵¹CrO₄) and aliquoted at a cell density of 5×10⁴ cells/well to each well of a 96 well plate. Purified binding proteins are added at a concentration of 10 μg/ml to the various dilutions of PBMCs. Spontaneous release is measured without addition of PBMC or binding protein, and maximal release is measured by the addition of detergent (1% NP-40) to the appropriate wells. Reactions are incubated and culture supernatant is harvested to a gamma scintillation counter (e.g., Lumaplate; Packard Instruments). Total and spontaneous lysis is determined by incubating target cells in 0.2% SDS or in complete medium, respectively. The percentage of lysis is calculated by the formula:

$$Lysis ( \% ) = \frac{Release in sample - spontaneous release }{Total lysis release - spontaneous release} \times 100$$

The percentage of lysis is expressed by LU that were determined by using the exponential fit equation described by Pross et al., J. Clin. Immunol. 1:51-63 (1981). One lytic unit is defined as the number of effector cells required to obtain 20% lysis of target cells.

Complement dependent cytotoxicity (CDC) assays may also be performed with ⁵¹Cr-labeled target cells in the presence of PBMCs (as described above for ADCC). Labeled cells are typically plated at 2000 cells/well in a 96-well plate containing increasing concentrations of binding protein and then incubated at 37° C. for 1 h with rabbit complement (Pei-Freez, Rogers, Ak.) at a final dilution of 1:100. Human sera from normal donors are added to the wells containing target cells and incubated at 37° C. Heat-inactivated serum may be used as a control to ensure measurement of complement-specific lysis. Specific target cell lysis is determined as described above for ADCC. Binding protein-mediated CDC is determined by subtracting the percentage of target cell lysis attributable to complement alone.

FcR binding may be assayed and quantified by assessing binding to soluble FcIg (e.g. CD64Ig, CD32Ig, CD16Ig, and CD89Ig) and/or on cells expressing the respective Fc receptor (i.e. CD64⁺, CD32⁺, CD16⁺, and CD89⁺ cells). FcR engagement and activation may, for example, be measured through the generation of superoxide by leucocytes (e.g., U937 cells).

### Contemplated Uses for Binding Proteins Having Altered Effector Function

Binding proteins of the present invention will find utility in a wide variety of therapeutic applications.

As described above, and as exemplified below, within certain embodiments, the present invention provides binding proteins comprising an insertion of one or more amino acids within an immunoglobulin hinge, CH2, and/or CH3 region, wherein the immunoglobulin exhibits an altered (i.e. an increased or decreased) binding affinity and/or specificity for one or more of FcγRI (CD64), FcγRII (CD32), and/or FcγRIII (CD16).

Contemplated uses for this class of binding protein include, for example, the targetted depletion of cell populations (a) in patients with low/hypofunctional natural killer (NK) cell populations and/or (b) in patients benefiting from the improved potency of the modified binding protein. Alternative contemplated uses for such binding proteins include the treatment of bacterial, parasitic, and/or viral infections wherein an increase or a decrease in binding to one or more of FcγRI, FcγRII, and/or FcγRIII increases pathogen neutralization or clearing. Alterations in such FcγR binding activity will also be useful for the treatment of infectious diseases wherein infection can be promoted by antibody FcγR interactions including, but not limited to, diseases such as HIV-1.

Within alternative embodiments are provided binding proteins that comprise an insertion of one or more N-linked and/or O-linked glycosylation sequencers) (such as, for example, one or more N-linked N-X-(S/T) glycosylation sequence(s) and/or one or more O-linked X-P-X-X (wherein at least one X is T), T-X-X-X (wherein at least one X is T), X-X-T-X (wherein at least one X is R or K), and S-X-X-X (wherein at least one X is S)) proximal to and/or distal to the site of N-linked and/or O-linked glycosylation in the corresponding native immunoglobulin Fc region, wherein the binding protein exhibits an altered (i.e. an increased or decreased) FcγR binding affinity and/or specificity, such as altered binding affinity and/or specificity for C1q, FcγRI (CD64), FcγRII (CD32), and/or FcγRIII (CD16).

Contemplated uses for this class of binding protein are, within certain aspects, based upon the preservation of the binding protein's half-life and corresponding reduction in cross-linking potential. For example, the present invention contemplates that such modified binding proteins will find use for the preferential targeting of one or more of C1q, FcγRI (CD64), FcγRII (CD32), and/or FcγRIII (CD16) with reduced cross-linking mediated intracellular signalling such as CD3 and/or CD28 signalling. Alternative contemplated uses for this class of binding protein include the preservation of half-life with lower potential for cell depletion and preservation of cross-linking for use when cross-linking drives desired signals but target cell depletion is not desired. Ex. Agonist SMIP for EPO-R.

Still further embodiments of the present invention provide binding proteins comprising the insertion and/or replacement of one or more amino acids within an IgG immunoglobulin CH2 and/or CH3 region wherein the amino acid insertion and/or replacement comprises one or more amino acids corresponding to an IgA immunoglobulin CH2 and/or CH3 region, wherein the one or more amino acid(s) of an IgA immunoglobulin CH2 and/or CH3 region participate in specific binding of an IgA immunoglobulin with its cognate Fcα receptor and wherein the modified binding protein is capable of specifically binding to FcαR.

Contemplated uses for this class of binding protein include targeted cell depletion wherein, for example, an binding protein comprising a combination of one or more IgG CH2 and/or CH3 region(s) and one or more IgA CH2 and/or CH3 region(s) is capable of binding to one or more of CD16, CD32, and/or CD64 as well as to CD89. Such binding proteins will enable the use of polymorphonuclear (PMN) effectors in addition to natural killer (NK)/monocyte effectors to achieve the elimination og target cells. Such alterations in the binding specificity for binding proteins described herein will, accordingly, result in improved potency and greater efficacy in a broad range of patient populations.

As noted above with respect to binding proteins comprising one or more inserted amino acid within an immunoglobulin hinge, CH2, and/or CH3 region, binding proteins having binding specificity for a combination of one or more IgG CH2 and/or CH3 region(s) and one or more IgA CH2 and/or CH3 region(s) will find use in the treatment of bacterial, parasitic, or viral infections where improvements in FcγR and/or in FcαR binding may result in an increase in pathogen neutralization and/or clearing activity. Alterations in FcγR and/or in FcαR binding will also be useful in the treatment of infectious diseases wherein infection can be promoted by antibody FcR interactions, such as diseases associated with HIV-1 infection.

The following Examples are offered by way of illustration, not limitation.

### EXAMPLES

### Example 1

### Modification of Loops within an Igg Immunoglobulin Hinge and/or CH2 Domains Confers Improved FcRγIII Binding Affinity

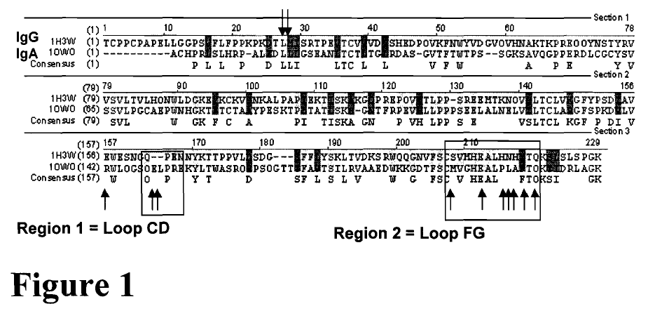

Member of the IgG class of antibodies specifically bind to CD16 (FcRγIII). Mutational changes within loop domains of an exemplary IgG antibody were constructed to alter the binding affinity of IgG for its cognate Fc receptor CD16. Specifically targeted were the four loops of the CH2 IgG domains that contact the CD16 molecule at two interfaces. Based upon the crystal structure described by Sondermann P. et al., Nature 406(6793):267-73 (2000), a first interface involves the interaction of CD16 with a hinge region loop and the FG loop of the alpha chain of CH2. A second interface involves an interaction of the CD16 molecule with the hinge region loop, BC loop, DE loop (i.e. a carbohydrate loop), and the FG loop of the beta chain of CH2. See FIG. 1 for a diagram of these contact sites.

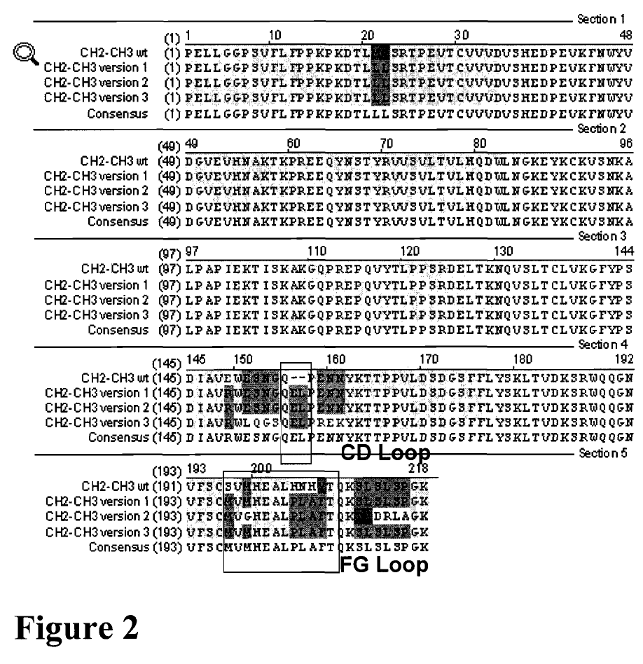

Insertion mutagenesis was employed to generate changes at the two interfaces within the following three non-carbohydrate loops: (1) the hinge region loop, (2) the BC loop, and (3) the FG loop. Libraries of such insertion mutations are suitable for selecting individual mutants having a desired binding affinity for one or more FcRγ receptor(s). The downward pointing arrows in FIG. 2 indicate representative locations for incorporating amino acid insertion(s) in order to achieve insertion mutants according to this aspect of the present invention.

Libraries of insertion mutants were constructed by inserting a polynucleotide sequence within the coding region for NWN sequences within each of the three hinge regions. Libraries 1A, 1B, and 1C were made at the hinge region loop, Libraries 2A and 2B were made at the BC loop, and Libraries 3A, 3B and 3C were made at the FG loop. Libraries containing 96 total members were constructed by inserting 12 unique sequences in each of 8 different positions. Amino acids having either long or bulky side chains: Phenylalanine (F), Leucine (L), Isoleucine (I), Methionine (M), Valine (V), Tyrosine (Y), Histidine (H), Glutamine (Q), Asparagine (N), Lysine (K), Aspartic acid (D), and Glutamic acid (E).

cDNA sequences for each of Libraries 1A, 1B, 1C, 2A, and 2B were constructed using an overlapping PCR extension method utilizing 6 oligonucleotide primers (sequences are provided in Table 3). Oligonucleotides for generating Library 1A are Lib1A_F1, Lib1A_F2, Lib1A_F3, Lib1A_R1, Lib1A_R2 and Lib1A_R3. Oligonucleotides for generating Library 1B, all the oligos are the same except oligonucleotide Lib1B_F2 replaces Lib1A_F2 and Lib1B_R1 replaces Lib1A_RI. Oligonucleotides for generating Lib1C, the oligos are the same again except oligo Lib1C_F2 replaces Lib1A_F2 and oligo Lib1C_R1 replaces Lib1A_R1. Oligonucleotides for generating Library 2A are Lib1A_F1, Lib2A_F2, Lib2A_F3, Lib2A_R1, Lib2A_R2 and Lib1A_R3. Oligonucleotides for generating Library 2B the oligonucleotides used are the same as for library 2A except that oligo Lib2B_F3 replaces Lib2A_F3 and oligonucleotides Lib2B_R2 replaces oligo Lib2A_R2.

For each library, 6 long oligonucleotide primers, at a concentration of 20 nM, were mixed with the two short end oligonucleotide primers (comprising restriction sites for Bci-I (forward primer) and Sac-II (reverse primer)) at a concentration of 1 μM. PCR reactions were set up using Invitrogen's supermix polymerase (Carlsbad, Calif.) employing the following conditions: (a) an initial 94° C. melting for 1 minute and (b) 30 cycles at 94° C. for 1 minute, 50° C. for 2 minutes, and 72° C. for 3 minutes.

TABLE 3

Oligonucleotide Primers for Generation of
Antibody Libraries 1A, 1B, 1C, 2A, and 2B

| | SEQ | |
|---|---|---|
| Oligo | ID | |
| Name | NO: | Sequence |
| Bci-I | 28 | ttcttctgatcaggagcccaaat |
| Forward | | |
| Sac-II | 29 | GCTCCTCCCGCGGCTTTGTCTTGG |
| Reverse | | |
| Lib1A_F1 | 30 | ttcttctgatcaggagcccaaatcttctgacaaaactca |
| | | cacatctccaccgtgcccag |
| Lib1A_F2 | 31 | gggaccgtcagtcttcctcttccccccaaaacccaagga |
| | | caccctcatgatctcccgga |
| Lib1A_F3 | 32 | tgtggtggacgtgagccacgaagaccctgaggtcaagtt |
| | | caactggtacgtggacggcg |
| Lib1A_R1 | 33 | AGAGGAAGACTGACGGTCCACCNWNCAAGAGTTCAGGTG |
| | | CTGGGCACGGTGGAGATGTGT |
| Lib1A_R2 | 34 | CGTGGCTCACGTCCACCACCACGCATGTGACCTCAGGGG |
| | | TCCGGGAGATCATGAGGGTGT |
| Lib1A_R3 | 35 | GCTCCTCCCGCGGCTTTGTCTTGGCATTATGCACCTCCA |
| | | CGCCGTCCACGTACCAGTTGA |
| Lib1B_F2 | 36 | wggaccgtcagtcttcctcttccccccaaaacccaagga |
| | | caccctcatgatctcccgga |
| Lib1B_R1 | 37 | AGAGGAAGACTGACGGTCCNWNACCCAAGAGTTCAGGTG |
| | | CTGGGCACGGTGGAGATGTGT |
| Lib1C_F2 | 38 | gnwnccgtcagtcttcctcttccccccaaaacccaagga |
| | | caccctcatgatctcccgga |
| Lib1C_R1 | 39 | AGAGGAAGACTGACGGNWNTCCACCCAAGAGTTCAGGTG |
| | | CTGGGCACGGTGGAGATGTGT |
| Lib2A_F2 | 40 | gccgtcagtcttcctcttccccccaaaacccaaggacac |
| | | cctcatgatctcccggaccc |
| Lib2A_F3 | 41 | tgtggacgtgnwnagccacgaagaccctgaggtcaagtt |
| | | caactggtacgtggacggcg |
| Lib2A_R1 | 42 | GGAAGAGGAAGACTGACGGTCCACCCAAGAGTTCAGGTG |
| | | CTGGGCACGGTGGAGATGTGT |
| Lib2A_R2 | 43 | CGTGGCTNWNCACGTCCACCACCACGCATGTGACCTCAG |
| | | GGGTCCGGGAGATCATGAGG |
| Lib2B_F3 | 44 | tgtggacgtgagcnwncacgaagaccctgaggtcaagtt |
| | | caactggtacgtggacggcg |
| Lib2B_R2 | 45 | CGTGNWNGCTCACGTCCACCACCACGCATGTGACCTCAG |
| | | GGGTCCGGGAGATCATGAGGG |

The amplified fragments were ligated into Invitrogen's TOPO vector and transformed into TOP10 bacterial cells. In excess of 200 colonies were pooled and the complexity of each library was determined by sequence analysis of 15 clones. The fragments were then digested with Bci-I and Sac-II and ligated into a Bci-I/Sac-II digested pD18 expression vector encoding an anti-CD20 small modular immunopharmaceutical product (SMIP™ product) and engineered to remove an extra Sac-II restriction site as well as engineered with a stop codon and a unique restriction site (Not-I) to permit linearization of the background vector containing the wild type CH2 domain.

The genes for libraries 3A, 3B, and 3C were constructed using the Quikchange method of Stratagene (La Jolla, Calif.). 33 base-pair sense and antisense oligonucleotide primers were designed to facilitate incorporation of nucleotide sequences encoding the amino acid sequence NWN (i.e. asparagine-tryptophan-asparagine). The sequences of these oligonucleotide primers are presented in Table 4 Oligonucleotides for Library 3A are Lib3A-F and Lib3A-R. Oligonucleotides for Library 3B are Lib3B-F and Lib3B-R. Oligonucleotides for Library 3C are Lib3C-F and Lib3C-R.

TABLE 4

Oligonucleotide Primers for Generation of
Antibody Libraries 3A, 3B and 3C

| | SEQ | |
|---|---|---|
| Oligo | ID | |
| Name | NO: | Sequence |
| lib3A-F | 46 | gtctccaacaaagccnwnctcccagcccccatc |
| lib3A-R | 47 | GATGGGGGCTGGGAGNWNGGCTTTGTTGGAGAC |
| Lib3B-F | 48 | cccaacaaagccctcnwnccagcccccatcgag |
| Lib3B-R | 49 | CTCGATGGGGGCTGGNWNGAGGGCTTTGTTGGAG |
| Lib3C-F | 50 | cacaaagccctcccanwngcccccatcgagaaaac |
| Lib3C-R | 51 | GTTTTCTCGATGGGGGCNWNTGGGAGGGCTTTGTTG |

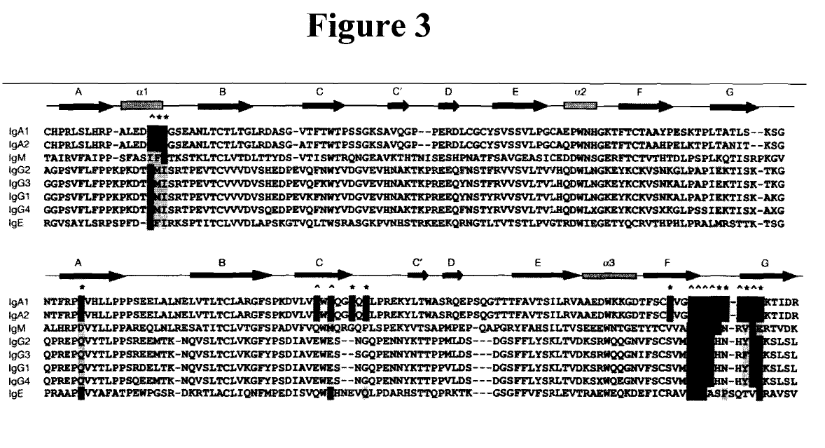

For each library, a 100 μL PCR reaction contained 20 ng of template DNA (i.e. a pD18 expression vector encoding a CD37-specific small modular immunopharmaceutical product (SMIP™ product), 125 ng each of the forward and reverse oligonucleotide primers, 500 nM dNTP, and 2.5 units of Stratagene's Ultra Pfu DNA. The following conditions were employed for the PCR reaction: (a) initial melting at 95° C. for 1 minute and (b) 18 cycles of 95° C. for 1 minute, 60° C. for 1 minute, and 68° C. for 6.5 minutes. Following each PCR reaction, wild-type vector was digested by incubating with the restriction enzyme Dpn-I for 2 hours. The DNA mixture was then transformed into TOP10 bacterial cells. In excess of 200 colonies from each library were pooled and the complexity of each library was determined by sequence analysis of 15 clones. The libraries were digested with Sac-II and Bsr-G1 restriction endonucleases and ligated into pD18 with an anti-CD37 front end, an extra Sac-II site, a stop codon, and a unique Not1 restriction site to linearize the background vector containing the wild type CH2 sequence. FIG. 3.

Member clones from each library were expressed in COS cells in 96-well or 24-well plates. Binding of clones to CD16 may be tested by employing a biotinylated CD16 with a human (HuIg) or murine (MuIg) immunoglobulin tail and screening individual candidate proteins by standard ELISA methodology wherein plates are coated with protein A and supernatants containing the individual candidates are added followed by CD16 HuIg-Biotin and streptavidin-HRP. Molecules possessing higher specific binding affinity for CD16 as compared to a corresponding wild-type SMIP™ product may then be selected for further characterization in an ADCC assay and for interaction with Protein A.

### Example 2

### Modification of Loops within an IgG Immunoglobulin CH3 Domain Confers Unique Binding Specificities

This Example discloses exemplary modifications within the IgG CH3 region that provide new, non-native recognition surfaces and, hence, binding specificities.

Several loops within the CH3 domain of IgG were engineered to provide IgA-specific binding interactions (referred to herein as IgG/A loopers). Expression of the following three IgG/A looper constructs were made and expressed in Cos cells: (a) IgG amino acids within the IgG CH3 domain were replaced with amino acids from the IgA CH3 domain, which amino acids in the wild-type IgA immunoglobulin directly contact the Fcα-receptor (i.e. CD89), as well as two additional amino acids within the CD loop; (b) IgG amino acids within the IgG CH3 FG loop domain were replaced with amino acids from the IgA CH3 FG loop domain as well as other amino acids that contact the Fcα-receptor; and (c) IgG amino acids within the IgG CH3 CD loop domain were replaced with amino acids from the CH3 CD loop domain as well as other amino acids that contact the Fcα-receptor. In all cases, the front end of each of the resulting mutant IgG SMIP™-products was humanized 2H7-018014.

The binding specificity and/or affinity of an FcRα-receptor binding site was modified by changing amino acid residues at the CD and FG loops of CH3. FIG. 1 shows the sequence alignment of IgG and IgA CH2 and CH3 domains. The tertiary structures of IgG and IgA are quite similar as indicated by the RMS deviation of 1.7 Å when the backbones are superimposed.

The three genes were constructed using an overlapping PCR extension methodology. For each version, a set of 4 long oligonucleotides at a concentration of 20 nM were mixed with the two short end oligonucleotides (short F and short R) at a concentration of 1 uM. PCR reactions were set up using Invitrogen's supermix polymerase employing the following conditions: (1) an initial 94° C. melting for 1 minute and (2) 30 cycles of the following: 94° C. for 1 minute, 50° C. for 2 minutes, and 72° C. for 3 minutes. The amplified fragments were digested with Bsr-G1 and Xba-I and inserted into a pD18 vector harboring a humanized anti-CD20 SMIP™-product (018014) digested with Bsr-G1 and Xba-I to remove the wild-type CH3 domain. Sequences for the oligonucleotides are presented in Table 5

TABLE 5

Oligonucleotide Primers for Modification of
Loops within an IgG Immunoglobulin CH3 Domain

| | SEQ | |
|---|---|---|
| Oligo | ID | |
| Name | NO: | Sequence |
| F1_ver1 | 52 | cagaaccacaggtgtacaccctgcccccatcccgggatg |
| | | agctgaccaagaaccagg |
| F2_ver1 | 53 | agcttctatccaagcgacatcgccgtgcgttgggagagc |
| | | aatgggcaggagctgccg |
| F3_ver1 | 54 | ccccgtgctggactccgacggctccttcttcctctacag |
| | | caagctcaccgtggacaa |
| F4-_ver1 | 55 | gcttctcctgcatggtgatgcatgaggctctgccactcg |
| | | ccttcacgcagaagagcc |
| R1_ver1 | 56 | tgcttggatagaagcctttgaccaggcaggtcaggctga |
| | | cctggttcttggtcagct |
| R2_ver1 | 57 | cgagtccagcacgggaggcgtggtcttgtagttgttctc |
| | | cggcagctcctgcccatt |
| R3_ver1 | 58 | cccatgcaggagaagacgttcccctgctgccacctgctc |
| | | ttgtccacggtgagcttg |
| R4_ver1 | 59 | cgctataatctagatcatttacccggagacagggagagg |
| | | ctcttctgcgtgaagg |
| short-F | 60 | cagaaccacaggtgtacaccctgccc |
| short-R | 61 | cctataatctagatcatttacc |
| F4_ver2 | 62 | gtcttctcctgcatggtgggccacgaggccctgccgctg |
| | | gccttcacacagaagacca |
| R4_ver2 | 63 | cgctataatctagatcatttacccgccaagcggtcgatg |
| | | gtcttctgtgtgaagg |
| F2_ver3 | 64 | aggcttctatccaagcgacatcgccgttcgctggctgca |
| | | ggggtcacaggagctgccc |
| R2_ver3 | 65 | cgagtccagcacgggaggcgtggtcttgtacttctcgcg |
| | | gggcagctcctgtgaccc |

Replacement of the MI amino acids to LL near the beginning of the CH2 region was performed by PCR mutagenesis. These two amino acids are additional contact points between immunoglobulins of the IgA isotype and their cognate Fc receptor CD89 (FcαR). See, FIG. 2. Sequences of each of the three modified IgG-based SMIP™-products were confirmed and the corresponding pD18 vectors were transfected into COS cells for gene expression. Supernatants were examined for binding activity to CD20 by staining Wi12S cells and flow cytometric analysis. CD20 staining with 2H7 SMIP CH3 mutated for binding to IgA receptor.

Supernatants were first incubated with Wi12S cells for 30 minutes on ice. Cells were washed twice and 1:100 dilution of PE conjugated anti-human IgG was added. Cells were washed again and MF for each sample was determined on the flow Cytometry. Each of the three modified immunoglobulins was expressed at a level of approximately 2-4 μg/mL when compared to the standard curve.

To test binding of these molecules to the IgA receptor, Wi12S cells were incubated with the supernatants for 30 minutes, washed twice with 1% BSA/PBS, and 5 μg/ml of CD89 (IgA receptor) human Ig in PBS was added and incubated for another 30 minutes. Cells were washed again and 50 μL of 1:100 diluted PE conjugated protein A in 1% BSA/PBS was added and incubated for 30 minutes. Samples were washed with 1% BSA/PBS and resuspended in 100 ml of the washing buffer. The samples were then read by flow cytometry.

Each of the three modified immunoglobulins was expressed at a level of approximately 2-4 μg/ml, purified using protein A affinity chromatography, and characterized for specific binding to Wi12S cells and to CD89 by conventional flow cytometric methodologies employing a CD89 HuIg and PE-labeled protein A.

What is claimed is:

1. A binding protein comprising one or more immunoglobulin constant region hinge, CH2, and/or CH3 domain(s) wherein said one or more hinge and/or constant region CH2 and/or CH3 domain comprises an insertion and/or a deletion of one or amino acids wherein said binding protein exhibits an altered binding affinity for one or more cognate Fc receptor.

2. The binding protein of claim 1 wherein said binding protein is selected from the group consisting of an antibody, an antibody fragment, and a small modular immunopharmaceutical products (SMIP™ products).

3. The binding protein of claim 1, said binding protein comprising one or more modified IgG immunoglobulin heavy chain hinge, CH2, and/or CH3 domain, wherein said one or more IgG immunoglobulin heavy chain hinge, CH2, and/or CH3 domain is modified by the insertion of one or more amino acid(s) and/or deletion of one or more amino acid(s), wherein said modified binding protein binds to an Fc receptor selected from the group consisting of FcγRI (CD64), FcγRII (CD32), and FcγRIII (CD16) with a higher affinity as compared to the corresponding binding protein comprising unmodified IgG immunoglobulin heavy chain hinge, CH2, and/or CH3 domains.

4. The binding protein of claim 3 wherein said modified IgG domain is a modified IgG₁ CH2 domain, a modified IgG₂ CH2 domain, a modified IgG₃ CH2 domain, and/or a modified IgG₄ CH2 domain.

5. The binding protein of claim 4 wherein said modified IgG domain is a modified IgG₁ CH2 domain and/or a modified IgG₃ CH2 domain and wherein said modified IgG₁ CH2 domain and/or modified IgG₃ CH2 domain comprises one or more amino acid deletion from and/or amino acid insertion within the hinge proximal loop structure L-L-G-G-P of the IgG₁ and/or IgG₃ CH2 domain.

6. The binding protein of claim 5 wherein said binding protein comprises one or more insertion(s) of one or more amino acid(s) within the hinge proximal loop structure L-L-G-G-P.

7. The binding protein of claim 6 wherein said hinge proximal loop structure comprises an insertion of one or more amino acid(s) at the position “*” such that the hinge proximal loop structure comprises a sequence selected from the group consisting of L-L-*-G-G-P, L-L-G-*-G-P, and L-L-G-G-*-P, wherein “*” indicates the insertion of at least 1, 2, 3, 4, 5, 6, 7, 8, 9, 10, 11, 12, 13, 14, 15, 16, 17, 18, 19, or 20 amino acids.

8. The binding protein of claim 7 wherein said “*” comprises one or more amino acid selected from the group consisting of Ala, Gly, Ile, Leu, and Val.

9. The binding protein of claim 4 wherein said modified IgG domain is a modified IgG₁ CH2 domain, a modified IgG₂ CH2, and/or a modified IgG₃ CH2 domain and wherein said modified IgG₁ CH2 domain, modified IgG₂ CH2, and/or modified IgG₃ CH2 domain comprises one or more amino acid deletion from and/or amino acid insertion within the BC loop structure D-V-S-H-E of the IgG₁, IgG₂, and/or IgG₃ CH2 domain.

10. The binding protein of claim 9 wherein said binding protein comprises one or more insertion(s) of one or more amino acid(s) within the BC loop structure D-V-S-H-E.

11. The binding protein of claim 10 wherein said BC loop structure comprises an insertion of one or more amino acid(s) at the position “*” such that the BC loop structure comprises a sequence selected from the group consisting of D-V-*-S-H-E and D-V-S-*-H-E, wherein “*” indicates the insertion of at least 1, 2, 3, 4, 5, 6, 7, 8, 9, 10, 11, 12, 13, 14, 15, 16, 17, 18, 19, or 20 amino acids.

12. The binding protein of claim 11 wherein said “*” comprises one or more amino acid selected from the group consisting of Ala, Gly, Ile, Leu, and Val.

13. The binding protein of claim 4 wherein said modified IgG domain is a modified IgG₁ CH2 domain and/or a modified IgG₃ CH2 domain, wherein said modified IgG₁ CH2 domain and/or a modified IgG₃ CH2 domain comprises one or more amino acid deletion from and/or amino acid insertion within the FG loop structure, A-L-P-A-P-I of the IgG₁ and/or IgG₃ CH2 domain.

14. The binding protein of claim 13 wherein said binding protein comprises one or more insertion(s) of one or more amino acid(s) within the FG loop structure A-L-P-A-P-I.

15. The binding protein of claim 14 wherein said FG loop structure comprises an insertion of one or more amino acid(s) at the position “*” such that the BC loop structure comprises a sequence selected from the group consisting of A-L-*-P-A-P-I, A-L-P-*-A-P-I, and A-L-P-A-*-P-I, wherein “*” indicates the insertion of at least 1, 2, 3, 4, 5, 6, 7, 8, 9, 10, 11, 12, 13, 14, 15, 16, 17, 18, 19, or 20 amino acids.

16. The binding protein of claim 15 wherein said “*” comprises one or more amino acid selected from the group consisting of Ala, Gly, Ile, Leu, and Val.

17. The binding protein of claim 1, said binding protein comprising one or more modified IgG heavy chain hinge, CH2, and/or CH3 domain, wherein said one or more heavy chain hinge, CH2, and/or CH3 domain is modified by the insertion of one or more N-linked glycosylation sequence(s) at one or more position that is proximal and/or distal to a native glycosylation sequence, which glycosylation sequence is sufficient to achieve N-linked glycosylation at the position of insertion, wherein said modified binding protein binds to an Fc receptor selected from the group consisting of FcγRI (CD64), FcγRII (CD32), and FcγRIII (CD16) with a higher affinity as compared to the corresponding binding protein comprising unmodified IgG immunoglobulin heavy chain hinge, CH2, and/or CH3 domains.

18. The binding protein of claim 17 wherein said hinge, CH2, and/or CH3 domain is an IgG₁ hinge, CH2, and/or CH3 domain, an IgG₂ hinge, CH2, and/or CH3 domain, an IgG₃ hinge, CH2, and/or CH3 domain, and/or an IgG₄ hinge, CH2, and/or CH3 domain comprising the insertion of one or more N-linked glycosylation sequence N-X-(S/T), wherein X is any amino acid proximal to and/or distal to a site of N-linked glycosylation in the corresponding native IgG immunoglobulin hinge, CH2, and/or CH3 domain.

19. The binding protein of claim 18 wherein said binding protein comprises an IgG CH2 domain wherein said CH2 domain comprises an insertion of one or more N-X-(S/T) sequence adjacent to and/or within 0 to 100 amino acids amino-terminal and/or carboxy-terminal to the native N-S-T sequence within the DE loop of said IgG CH2 domain.

20. The binding protein of claim 19 wherein said IgG CH2 domain comprises the amino acid sequence N-S-T inserted adjacent to and/or within 0 to 100 amino acids amino-terminal and/or carboxy-terminal to the native N-S-T sequence such that the native amino acid sequence X-N-S-T-Z is modified to (AA^{a})-N-S-T-(AA^{b})-N-S-T-(AA^{c}) wherein each of AA^{a} AA^{b}, and AA^{c} independently designate from 1 to 100 amino acids.

21. The binding protein of claim 20 wherein said IgG CH2 domain is modified such that the amino acid sequence N-S-T is inserted within one amino acid from the native N-S-T sequence such that the native amino acid sequence X-N-S-T-Z is modified to X-N-S-T-Z-N-S-T-Z, wherein X and Z are independently selected from Tyr (Y) and Phe (F).

22. The binding protein of claim 18 wherein said N-linked glycosylation sequence is inserted within the BC loop of one or more IgG₁, IgG₂, IgG₃, and/or IgG₄ CH3 domain distal to said native site of N-linked glycosylation within the CH2 domain such that the native amino acid sequence Y-P-S-D-I-A is modified to Y-P-N-S-T-D-I-A or to Y-N-S-T-P-S-D-I-A.

23. The binding protein of claim 3 wherein said binding protein comprises one or more heavy chain hinge, CH2, and/or CH3 domain of a first immunoglobulin class selected from IgA, IgD, IgE, IgG, and IgM, wherein the binding protein is modified by amino acid replacement and/or amino acid insertion in the primary amino sequence of said one or more heavy chain hinge, CH2, and/or CH3 domain of the first immunoglobulin class to generate a binding protein capable of binding to one or more Fc receptor of a second immunoglobulin class, wherein said second immunoglobulin class is distinct from said first immunoglobulin class.

24. The binding protein of claim 23 wherein said first immunoglobulin class is IgG and wherein said second immunoglobulin class is IgA.

25. The binding protein of claim 24 wherein said binding protein is capable of specifically binding (a) to an Fc receptor selected from the group consisting of FcγRI (CD64), FcγRII (CD32), and FcγRIII (CD16) and (b) to FcαR (CD89).

26. The binding protein of claim 25 wherein said binding protein comprises one or more amino acid substitution(s) within the IgG CH3 FG loop and/or one or more amino acid substitution(s) within the IgG CH3 CD loop.

27. The binding protein of claim 26 wherein said binding protein comprises the replacement of the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof.

28. The binding protein of claim 27 wherein said binding protein comprises the replacement of the IgG CH3 CD loop comprising the amino acid sequence Q-P-E-N, or a portion thereof, with the IgA CH3 CD loop comprising the amino acid sequence Q-E-L-P-R-E, or a portion thereof.

29. The binding protein of claim 28 wherein said binding protein comprises the replacement of both the IgG CH3 FG loop comprising the amino acid sequence C-S-V-M-H-E-A-L-H-N-H-Y-T-Q, or a portion thereof, with the IgA CH3 FG loop comprising the amino acid sequence C-M-V-G-H-E-A-L-P-L-A-F-T-Q, or a corresponding portion thereof, and the IgG CH3 CD loop comprising the amino acid sequence Q-P-E-N, or a portion thereof, with the IgA CH3 CD loop comprising the amino acid sequence Q-E-L-P-R-E, or a portion thereof.

30. The binding protein of any one of claims 27-29 further comprising the substitution of IgG heavy chain CH3 amino acid Met at CH3 amino acid position no. 28 within the sequence K-D-T-L-M-I-S-R-T with amino acid Leu such that the binding protein further comprises the amino acid sequence K-D-T-L-L-I-S-R-T.

31. The binding protein of any one of claims 27-29 further comprising the substitution of IgG heavy chain CH3 amino acid Glu at CH3 amino acid position no. 157 within the sequence D-I-A-V-E-W-E-S-N with amino acid Arg such that the binding protein further comprises the amino acid sequence D-I-A-V-R-W-E-S-N.
